(19) United States
(12) Patent Application Publication
(10) Pub. No.: US 2015/0079113 A1
(43) Pub. Date: Mar. 19, 2015

(54) MENA AND ALPHA5 INTEGRIN INTERACTION

(71) Applicant: MASSACHUSETTS INSTITUTE OF TECHNOLOGY, Cambridge, MA (US)

(72) Inventors: Frank B. Gertler, Boston, MA (US); Stephanie Gupton, Chapel Hill, NC (US); Daisy Riquelme, Somerville, MA (US); Shannon K. Hughes-Alford, Cambridge, MA (US); Madeleine J. Oudin, Cambridge, MA (US)

(73) Assignee: MASSACHUSETTS INSTITUTE OF TECHNOLOGY, Cambridge, MA (US)

(21) Appl. No.: 14/395,617

(22) PCT Filed: May 9, 2013
(86) PCT No.: PCT/US13/40274

§ 371 Date: Oct. 20, 2014

Related U.S. Application Data
(60) Provisional application No. 61/788,411, filed on Mar. 15, 2013.
(60) Provisional application No. 61/645,782, filed on May 11, 2012.

(51) Int. Cl. C07K 16/18; C12N 15/115; G01N 33/50; C07K 2/00

(52) U.S. Cl. CPC C07K 16/18; C07K 2/00; C12N 15/115; G01N 33/5011; G01N 2500/02; C12N 2310/16
USPC 424/174.1; 514/19.3; 514/44 R; 435/7.1

(57) ABSTRACT

Methods are provided for treating invasion of a tumor or metastasis of a tumor in a subject comprising administering to the subject an agent which inhibits the interaction of Mena with an alpha5 integrin. Assays for identifying agents that inhibit interaction of Mena with an alpha5 integrin are also provided.

### CROSS-REFERENCE TO RELATED APPLICATIONS

This application claims benefit of U.S. Provisional Application No. 61/645,782, filed May 11, 2012, and of U.S. Provisional Application No. 61/788,411, filed Mar. 15, 2013, the contents of each of which are hereby incorporated by reference.

### STATEMENT OF GOVERNMENT SUPPORT

This invention was made with Government support under Grant Nos. R01 GM58801 and U54-CA112967 awarded by the National Institutes of Health. The Government has certain rights in this invention.

### BACKGROUND OF THE INVENTION

Throughout this application various publications, books, patents and patent application publications are referred to. The disclosures of all of these are hereby incorporated by reference in their entireties into the subject application to more fully describe the art to which the subject application pertains.

The extracellular matrix (ECM) is a three dimensional network of proteins secreted, assembled and remodeled dynamically by cells that it contacts (Hynes and Naba, 2012; Wickstrom et al., 2011). Cell migration, differentiation and other processes are controlled by the ECM as it engages adhesion receptors and presents matrix-bound growth factors to their cell surface receptors. One of the best-characterized ECM proteins is fibronectin (FN), an abundant, ubiquitous component of the interstitial matrix (Singh et al., 2010). Outside of the bloodstream, FN typically functions in multimeric fibrils assembled by cells from soluble FN dimers and organized into complex meshworks (Schwarzbauer and DeSimone, 2011). These elaborate FN matrices surround and connect cells, providing a supporting scaffold capable of delivering complex sets of multivalent, spatially organized biochemical and mechanical signals that influence many aspects of cell behavior (Hynes, 2009; Huttenlocher and Horwitz, 2011; Geiger and Yamada, 2011).

The predominant ECM receptors proteins are integrins, a family of heterodimeric transmembrane proteins comprised of α and β subunits that link the ECM to the cytoskeleton and transmit signals and mechanical forces bi-directionally across the plasma membrane (Hynes, 2002). Integrins are regulated by clustering and conformational changes triggered either “outside in” by binding to their specific ECM ligands, or “inside out” by interaction between the intracellular tails of integrin subunits and cytoplasmic proteins (Margadant et al., 2011). The β subunit cytoplasmic tails share significant sequence similarity; several cytoplasmic proteins directly bind most β subunits to regulate integrin activation, trafficking and signaling (Moser et al., 2009; Calderwood, 2004). In contrast, the a integrin subunit tails share only a short, conserved membrane-proximal sequence that interacts directly with the β subunit and with proteins that regulate integrin trafficking (Ivaska and Heino, 2011) and with Sharpin, a negative regulator of integrin activation (Rantala et al., 2011). Less is known, however, about the potential unique functions conferred by the distal, divergent cytoplasmic tails of the 18 α subunits.

The two major FN receptors are αVβ3 and α5β1 (Hynes, 2002). α5β1 is the primary receptor for soluble FN and plays the predominant role in assembling FN into fibrils, though αVβ3 can assemble fibrils in cells lacking α5β1 (Yang et al., 1999). While αVβ3 and α5β1 can substitute for one another partially, typically they exert distinct effects on cell motility, invasion, signaling and matrix remodeling (Clark et al., 2005; Wickstrom et al., 2011; Caswell et al., 2009). For example, αVβ3 suppresses recycling of the epidermal growth factor receptor (EGFR), while inhibition or absence of αVβ3 drives α5β1 into a protein complex with EGFR mediated by Rab coupling protein (RCP) that drives coordinate recycling of the two receptors, dysregulates their signaling and promotes tumor cell invasion (Caswell et al., 2008; Muller et al., 2009).

Integrin-based ECM adhesions are dynamic, complex structures that turn over continually while changing their composition and morphology (Geiger and Yamada, 2011). Typically, new adhesions form as small integrin-rich punctae near the leading edge of spreading or migrating cells with associated cytoplasmic proteins bound to integrin tails that recruit additional signaling, adaptor or actin-binding proteins (Vicente-Manzanares and Horwitz, 2011). Nascent adhesions enlarge into focal complexes (FXs), more elongated, transient structures that mature into focal adhesions (FAs), larger structures that vary in composition and size that connect to the distal ends of Factin bundles. In some cell types, including fibroblasts, α5β1 exits from FAs and moves toward the cell interior along stress fibers (Pankov et al., 2000) into mature fibrillar adhesions (FBs), stable internal adhesions that mediate the critical process of FN fibrillogenesis. FBs are enriched for FN, α5β1 and tensin, the latter of which is not found in FXs and only weakly in FAs (Zaidel-Bar et al., 2007; Pankov et al., 2000; Zamir et al., 2000). FBs lack many abundant FA components, including phosphotyrosine-containing proteins, vinculin, FAK and zyxin. Fibrillogenesis begins as α5β1 translocates bound to FN out of FAs to FBs. This movement generates contractile forces on the α5β1-connection between the cytoskeleton and FN leading to conformational changes in α5β1 that strengthen and prolong FN binding (Margadant et al., 2011). The tensile forces also drive conformational changes in FN that expose self-association sites and align the nascent FN fibrils with intracellular actin bundles (Schwarzbauer and DeSimone, 2011).

The Ena/VASP family of actin-regulatory proteins plays diverse roles in cell movement and morphogenesis (Drees and Gertler, 2008; Bear and Gertler, 2009; Homem and Peifer, 2009). Ena/VASP influences membrane protrusion dynamics by promoting formation of longer, less-branched F-actin networks. Ena/VASP proteins increase F-actin elongation rates by promoting transfer of actin monomer from profilin to free barbed ends while protecting growing filaments from capping proteins that terminate polymerization (Hansen and Mullins, 2010; Bear and Gertler, 2009; Dominguez, 2009). Ena/VASP proteins are concentrated in sites of rapid actin assembly such as the tips of lamellipodia and filopodia. They also localize prominently to cell:cell and cell:matrix adhesions and interact with several FA components, including vinculin, zyxin, RIAM and palladin (Pula and Krause, 2008). While the function of Ena/VASP in FAs is not well understood, they are known to regulate integrin activation. For example, VASP negatively regulates αIIbβ3 activation in platelets (Aszodi et al., 1999; Hauser et al., 1999).

The three vertebrate Ena/VASP proteins Mena, VASP, and EVL share conserved domains (Gertler et al., 1996), including: 1) an N-terminal EVH1 domain that binds to proteins that typically contain one or more EVH1-binding sites with an optimal core motif of “FPPPP” (FP4) (Ball et al., 2002), though unconventional EVH1 ligands have been identified (Boeda et al., 2007); 2) a proline-rich center containing binding sites for SH3- and WW-domains, and the actin-monomer binding protein profilin (Ferron et al., 2007); 3) a C-terminal EVH2 domain that contains both G and F-actin binding sites and a coiled-coil that mediates their tetramerization (Barzik et al., 2005; Zimmermann et al., 2002) (FIG. 3A). Given their similarity, it is not surprising that expression of any of the three proteins is sufficient to support many Ena/VASP-dependent cellular functions such as filopodia) formation and extension (Applewhite et al., 2007; Dent et al., 2007), formation of functional endothelial barriers (Furman et al., 2007), or stimulating the actin-based motility of the intracellular pathogen Listeria monocytogenes (Geese et al., 2002). Recently, however, paralog-specific functions for Ena/VASP have been reported.

For example, a Mena isoform produced by alternate splicing, Mena^{INV} (Gertler and Condeelis, 2011), promotes carcinoma metastasis by potentiating chemotactic responses to EGF (Roussos et al., 2011a; Philippar et al., 2008); but neither VASP nor EVL produce isoforms equivalent to Mena^{INV}. Mena also has a unique low-complexity region of unknown function containing 13 repeats of α5-residue motif within a 91-residue span, termed the LERER-repeat (Gertler et al., 1996) (FIG. 3A, B).

The present invention provides novel treatments and assays based on the discovery of the interaction of Mena with integrins as disclosed hereinbelow.

### SUMMARY OF THE INVENTION

A method is provided of treating invasion of a tumor in a subject or inhibiting metastasis of a tumor in a subject comprising administering to the subject an agent which inhibits the interaction of Mena with an alpha5 integrin in an amount effective to treat invasion or inhibit metastasis of a tumor.

A method is also provided of treating a fibronectin deposition disease in a subject or a fibroproliferative disease in a subject comprising administering to the subject an agent which inhibits the interaction of Mena with an alpha5 integrin in an amount effective to treat fibronectin deposition or fibroproliferative disease.

A method for identifying an agent as an inhibitor of an interaction of Mena with an alpha5 integrin, the method comprising contacting the alpha5 integrin with Mena (a) in the presence of and (b) in the absence of the agent under conditions permitting Mena to interact with the alpha5 integrin and quantifying the interaction of Mena with the alpha5 integrin in the presence and in the absence of the agent, and identifying the agent as an inhibitor or not of an interaction of Mena with an alpha5 integrin, wherein quantification of a decreased interaction of Mena with the alpha5 integrin in the presence of the agent compared to in the absence of the agent indicates that the agent is an inhibitor of the interaction of Mena with the alpha5 integrin, and wherein quantification of no change in interaction, or an increased interaction, of Mena with the alpha5 integrin in the presence of the agent compared to in the absence of the agent indicates that the agent is not an inhibitor of the interaction of Mena with the alpha5 integrin.

Additional objects of the invention will be apparent from the description which follows.

### BRIEF DESCRIPTION OF THE FIGURES

FIG. 1A-1B: Expression of FP4-Mito recruits α5 integrin to the mitochondrial surface. A) Distribution of anti-α5 staining (middle) in wild-type primary meningeal fibroblasts (top row) or cells expressing FP4-Mito (right side) and stained for α5 (second row). Phalloidin staining shows F-actin distribution (left side). Bar is 10 μm. (B) MVD7 cells expressing GFP-Mena transiently transfected with mCherry-FP4-Mito (bottom panel in each row) and stained for the indicated proteins (top panel in each row). Bar is 10 μm.

FIG. 2A-2B: Mena associates with α5 and recruits it to FP4Mito-decorated mitochondria. MVD7 cells expressing mCherry-FP4Mito alone or with GFP-tagged Mena, VASP or EVL were stained with antibodies to α5 integrin and imaged. Arrowheads are a fiduciary mark for mCherry-FP4-Mito in MVD7 cells (top row). Arrows mark mCherry FP4-Mito in MVD7+GFP-Mena cells (second row). Bar is 10 μm. (B) Western blot analyses of α5 integrin immunoprecipitates from NIH3T3 cell lysates probed with antibodies to α5 integrin, β1 integrin, Mena, Paxillin and the p34 subunit of Arp2/3. “Lysate” represents 5% of the total protein used for immunoprecipitation; “IgG” is non-immune control antibody. Extraneous lanes were removed (white gaps).

FIGS. 3A-3D: The LERER repeat region of Mena is required for the interaction with α5 integrin. (A) Ena/VASP domains. (B) Sequence motif schematic for the LERER repeats in Mena; summed height of amino acids represents information content at that position; relative heights each residue are proportional to their usage in the given position. (C) α5 recruitment to mitochondria in MVD7 cells expressing the indicated GFP-tagged Mena deletion mutants and mCherry-FP4-Mito. (D) Anti-α5 immunoprecipitates from lysates of MVD7+GFP-Mena and MVD7+GFP-MenaΔLERER cells analyzed by western blot probed with antibodies to α5 or to GFP. Input represents 5% lysate used for immunoprecipitation; “α5 dpl” is 5% of the supernatant sampled after α5 immunoprecipitation.

FIG. 4A-4D: The LERER repeat region binds to, and localizes with α5 integrin. (A) MVD7 cells expressing mCherry-Mena (top row) and parental MVD7 cells (bottom row) expressing GFP-tagged LERER repeat. Bottom inset in both rows shows a region from the cell periphery; top inset shows a region from the cell center. Bar is 10 μm. (B) Western blot analysis of GST “pull down” binding assay with purified proteins. Purified GST and GST-α5 cytoplasmic tail were incubated with His tagged LERER or His-LERER-6×Glycine linker-Coiled Coil of Mena (His-LERER-CoCo) and analyzed by Western blot probed with anti-His antibodies. (“sup”, supernatant; “PD” pulled down). (C) Cells expressing FP4-Mito and α5-GFP Immunofluorescence staining for endogenous α5 integrin (Top). Arrows are fiduciary markers for GFP-FP4-Mito; Arrowheads indicate α5-GFP positive FAs (Bottom). (D) Pull-down binding assay using α5 tail lacking the C-terminal amino acids (GST-α5 tailΔCOOH).

FIG. 5A-5D: Distribution of α5 integrin to central FBs requires Mena. (A) MVD7 cells (top row) or MVD7 cells expressing GFP-Mena (middle row) or GFP-MenaΔLERER (bottom row) plated for 8 hours on FN-coated coverslips and stained for α5 and paxillin. Arrow indicates central region typically containing FBs. Arrowhead indicates a peripheral paxillin containing focal adhesion. Bar is 10 μm. (B) Average fraction of total cell area containing α5- or paxillin-positive ventral adhesions in MVD7, GFP-Mena, and MenaΔLERER-expressing cells (p<0.01, ANOVA LSD) (C) Quantification of data from FIG. 4A, percentage of ventral cell area containing α5-positive adhesions in cells expressing GFP-LERER-expressing or parental MVD7 cells, (p<0.01 t-test). (D) Rat2 fibroblasts were transfected with GFP-tensin, fixed, and stained for Mena. Scale bar is 15 μm.

FIG. 6A-6D: Expression and distribution of Mena and α5 integrin in primary cells lacking either protein. (A) Western blots of lysates from primary fibroblasts isolated from Mena^{FLOXED} (MenaF) also homozygous for a VASP deletion) or α5^{FLOXED} (α5F) mice 48 hrs after infection with GFP- or GFP-Cre adenovirus probed with antibodies to α5, Mena, VASP or tubulin as indicated. (B) qPCR analysis of Mena mRNA levels in α5F and α5 null fibroblasts Immunofluorescence of MenaF (C) or α5F (D) cells after infection with GFP- or GFP-CRE adenovirus as indicated.

FIG. 7A-7D: The Mena:α5 complex is enriched during cell spreading, (A) Anti-α5 integrin immunoprecipitates from lysates of MVD7+GFP-Mena cells in steady-state culture, suspension, or 30 minutes after plating were analyzed by western blot probed with antibodies as indicated. (B) Area of MVD7, MVD7+GFP-Mena, MVD7-GFP-cells 30 minutes after plating on FN-coated coverslips. (p<0.01, ANOVA LSD). (C) Examples of FRAP on MVD7 cells expressing either GFP-Mena or GFP-MenaΔLERER 30 minutes after plating on FN-coated coverslips. Fluorescence was photobleached (rectangle) and the recovery imaged over indicated time (s). (D) The t1/2 recovery of mCherry-zyxin or GFP-Mena of cells plated for 30 minutes on FN or Laminin (LN) (p<0.01, ANOVA LSD). (E) Percentage of total fluorescence recovery after photobleaching (ANOVA).

FIG. 8A-8C: Mena:α5 integrin interaction is necessary for normal fibrillogenesis. (A) MVD7 cells and MVD7 cells expressing GFP-Mena, GFP-MenaΔLERER, or GFP-VASP plated on vitronectin coated coverslips overnight and incubated with 10 μg/ml of fluorescently tagged FN for four hours prior to fixation and stained with anti-α5 antibodies. (B) Percentage of cell area containing FN fibrils (p<<0.01, ANOVA LSD). (C) Total amount of FN within fibrils per cell (p<<0.01, ANOVA LSD).

FIG. 9A-9B: Rescue of MVD7 hypermotility requires Mena capable of binding α5. (A) Wind-Rose plots of MVD7,MVD7+GFP-Mena or GFP-MenaΔLERER cell tracks over a six hour period. (B) Speed of indicated cells on FN for 6 hours.

FIG. 10A-10E: The LERER repeat region binds α5 integrin. (A) Purified GST or GST-α5 integrin cytoplasmic tail were incubated with His-LERER-EVH2 or His-EVH2 and the bound fraction analyzed by western blot with indicated antibodies. “PD”=pulled down. (B) Coomassie-stained gel of purified proteins as indicated. (C) and (D) Coomassie-stained gels of purified proteins used in binding assays shown in FIG. 4. (E) Plots of Paircoils2 analysis of LERER repeat. X axis is the position along the 91 residue LERER repeat. The Y axis indicates the probability that the structure is coiled-coil, p<0.025 is the cutoff for predicted coiled-coils.

FIG. 11: 2D Haptotaxis—D7 fibroblasts. Mena null fibroblasts do not move up a fibronectin gradient (FN). Expression of Mena Rescues this phenotype.

FIG. 12: MDA-MB-231 cells—3D Haptotaxis. FN gradient: 250 μg/ml Cells plated in 1 mg/ml collagen HIGH.

FIG. 13: Graphical representation of forward migration of WT-231 cells and Mena^{INV} positive cells in FN+EGF, low collagen. A FMI close to 1 indicates migration in the direction of gradient and zero indicates random. Expression of Mena^{INV} in MDA-MB-231 cells increases haptotaxis on a FN gradient in 3D

### DETAILED DESCRIPTION OF THE INVENTION

A fibronectin deposition disease is a disease which has symptoms or pathologies involving abnormal fibronectin deposition, for example a fibronectin glomerulopathy.

A fibroproliferative disease is a disease characterized by excessive accumulation of connective material in a critical location, such as fibroproliferative cardiovascular disease, pulmonary fibrosis, progressive kidney disease, systemic sclerosis, liver cirrhosis and fibroproliferative inflammatory bowel disease.

A method is provided of treating invasion of a tumor or inhibiting metastasis of a tumor in a subject comprising administering to the subject an agent which inhibits the interaction of Mena with an alpha5 integrin in an amount effective to treat invasion or inhibit metastasis of a tumor.

A method is also provided of treating a fibronectin deposition disease or a fibroproliferative disease in a subject comprising administering to the subject an agent which inhibits the interaction of Mena with an alpha5 integrin in an amount effective to treat fibronectin deposition or fibroproliferative disease.

In an embodiment, the agent inhibits the interaction of Mena with the C-terminal 5 residues of the alpha5 integrin C-terminal cytoplasmic tail. In an embodiment, the agent inhibits the interaction of a LERER repeat region of Mena with the alpha5 integrin. In an embodiment, the tumor is a breast cancer tumor. In an embodiment, the alpha5 integrin is part of an alpha5 beta1 integrin complex. In an embodiment, the alpha5 beta1 integrin is a fibronectin receptor. In an embodiment, the agent is a small organic molecule, an antibody, a fragment of an antibody, a peptide or an oligonucleotide aptamer. In an embodiment, the agent competes for binding to the alpha5 integrin with a LERER repeat region of Mena. In an embodiment, the Mena is human Mena. In an embodiment, the Mena is Mena^{INV}. In an embodiment, the Mena^{INV} is human Mena^{INV}.

A method for identifying an agent as an inhibitor of an interaction of Mena with an alpha5 integrin, the method comprising contacting the alpha5 integrin with Mena (a) in the presence of and (b) in the absence of the agent under conditions permitting Mena to interact with the alpha5 integrin and quantifying the interaction of Mena with the alpha5 integrin in the presence and in the absence of the agent, and identifying the agent as an inhibitor or not of an interaction of Mena with an alpha5 integrin, wherein quantification of a decreased interaction of Mena with the alpha5 integrin in the presence of the agent compared to in the absence of the agent indicates that the agent is an inhibitor of the interaction of Mena with the alpha5 integrin, and wherein quantification of no change in interaction, or an increased interaction, of Mena with the alpha5 integrin in the presence of the agent compared to in the absence of the agent indicates that the agent is not an inhibitor of the interaction of Mena with the alpha5 integrin.

In an embodiment, quantifying the interaction of Mena with the alpha5 integrin in the presence of and in the absence of the agent comprises quantifying the amount of Mena bound to alpha5 integrin. In an embodiment, quantifying the interaction of Mena with the alpha5 integrin in the presence and in the absence of the agent comprises quantifying the activity of alpha5 integrin. In an embodiment, the alpha5 integrin is part of an alpha5 beta1 integrin complex. In an embodiment, the agent is a small organic molecule, an antibody, a fragment of an antibody, a peptide or an oligonucleotide aptamer.

Assay techniques for use in the methods of the invention can comprise, in non-limiting examples, immunoprecipitation, protein purification, blots, and/or proximity ligation assays.

In an embodiment, the agent inhibits the interaction of Mena with the C-terminal 5 residues of the alpha5 integrin C-terminal cytoplasmic tail. In an embodiment, the agent is based on a LERER repeat region of Mena. In an embodiment, the agent is based on a LERER repeat region of Mena. In an embodiment, the agent competes for binding to the alpha5 integrin with a LERER repeat region of Mena. In an embodiment, the agent comprises a peptide having the sequence of the C-terminal 5 residues of the alpha5 integrin C-terminal cytoplasmic tail. In an embodiment, the Mena is human Mena. In an embodiment, the Mena is Mena^{INV}. In an embodiment, the Mena^{INV} is human Mena^{INV}.

A method is also provided for identifying an agent that binds to the LERER repeat region of Mena, the method comprising contacting the alpha5 integrin with Mena in the presence of an agent under conditions permitting Mena to interact with the LERER repeat region of alpha5 integrin and quantifying the interaction of Mena with the alpha5 integrin, wherein an agent that binds is identified as an agent that binds the LERER repeat region of alpha5 integrin, and wherein an agent that does not bind is identified as an agent that does not bind the LERER repeat region of alpha5 integrin.

In an embodiment of the methods disclosed herein, the Mena is a human Mena. In an embodiment, the Mena has the sequence set forth in Uniprot Q8N8S7. In another embodiment, the Mena is Mena^{INV}. In a further embodiment, the Mena^{INV} is human Mena^{INV}. In an embodiment, the Mena^{INV} is encoded by a Mena gene encoded mRNA but which contains the +++ exon and lacks the 11a exon.

In an embodiment of the methods disclosed herein, the alpha5 integrin is a human alpha5 integrin. In a further embodiment, the alpha5 beta1 integrin is human alpha5 beta1 integrin.

As used herein, “treating” a invasion of a tumor means that one or more symptoms of the invasion are inhibited, reduced, ameliorated, prevented, placed in a state of remission, or maintained in a state of remission. As used herein, “inhibiting” metastasis of a tumor in a subject” means that one or more symptoms or one or more other parameters by which the disease is characterized, are reduced, ameliorated, or prevented. Non-limiting examples of such parameters include uncontrolled degradation of the basement membrane and proximal extracellular matrix, and travel of tumor cells through the bloodstream or lymphatics, invasion, dysregulated adhesion, and proliferation at secondary site.

In an embodiment of the methods disclosed herein, the agent is a small organic molecule of 2000 daltons or less, an antibody, an antibody fragment, a peptide, a fusion protein or peptide, an RNAi agent or an oligonucleotide aptamer. In an embodiment of the methods disclosed herein, the agent is an RNAi agent and is an siRNA or a shRNA.

In an embodiment of the methods disclosed herein, the tumor is a mammary tumor. In an embodiment, the tumor is a tumor of a nasopharynx, pharynx, lung, bone, brain, sialaden, stomach, esophagus, testes, ovary, uterus, endometrium, liver, small intestine, appendix, colon, rectum, gall bladder, pancreas, kidney, urinary bladder, breast, cervix, vagina, vulva, prostate, thyroid or skin, or is a glioma.

As used herein a “small organic molecule” is an organic compound which contains carbon-carbon bonds, and has a molecular weight of less than 2000. The small organic molecule may also comprise inorganic atoms. The small molecule may be a substituted hydrocarbon or an substituted hydrocarbon. In an embodiment, the small molecule has a molecular weight of less than 1500. In an embodiment, the small molecule has a molecular weight of less than 1000.

As used herein “under conditions permitting Mena to interact with the alpha5 integrin” means conditions, for example as described herein, that permit Mena to interact with the alpha5 integrin excepting the presence of the tested agent.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Details

Here it is disclosed that the Mena LERER repeat interacts directly with the cytoplasmic tail of α5 integrin, mediating a robust adhesion-modulated interaction between Mena and α5β1. The Mena: α5 interaction contributes to key α5β1 functions that include FN fibrillogenesis, cell spreading and motility. Given their established roles in EGFR signaling responses, tumor cell motility, invasion and metastasis, a direct link between Mena and α5β1 is understood to play an important role in tumor-cell invasion and metastasis.

Relocalization of Mena to mitochondrial also recruits α5. While investigating Ena/VASP- and integrin-mediated neuritogenesis a serendipitous observation was made that the subcellular distribution of α5β1 could be influenced by Ena/VASP. A strategy was used to block Ena/VASP function by depleting them from their normal locations and sequestering them on the mitochondrial surface by expressing a construct containing EVH1-binding sites (FPPPP; “FP4”) fused to a mitochondrial-targeting motif (FP4-Mito)(Bear et al., 2000). FP4-Mito expression phenocopies defects arising from loss of Ena/VASP function in fibroblasts, endothelial cells, neurons and in Drosophila, where transgenic expression of FP4-Mito phenocopies axon-guidance and epithelial defects observed in Ena mutants (Bear et al., 2002; Furman et al., 2007; Dent et al., 2007; Gates et al., 2007). While FP4-Mito redistributes Ena/VASP proteins to the mitochondrial surface, it has no detectable effects on localization Ena/VASP-binding partners such as the FA proteins zyxin and vinculin, and causes no evident defects when expressed in Ena/VASP-deficient fibroblasts (Bear et al., 2000).

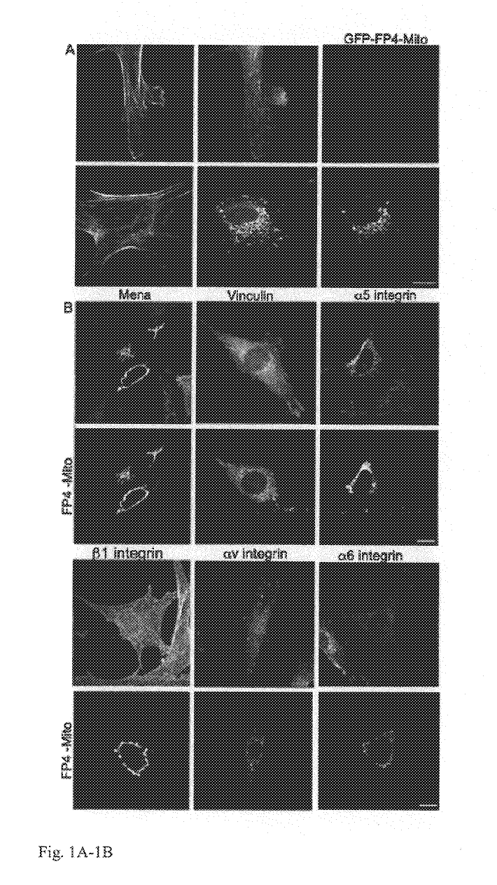

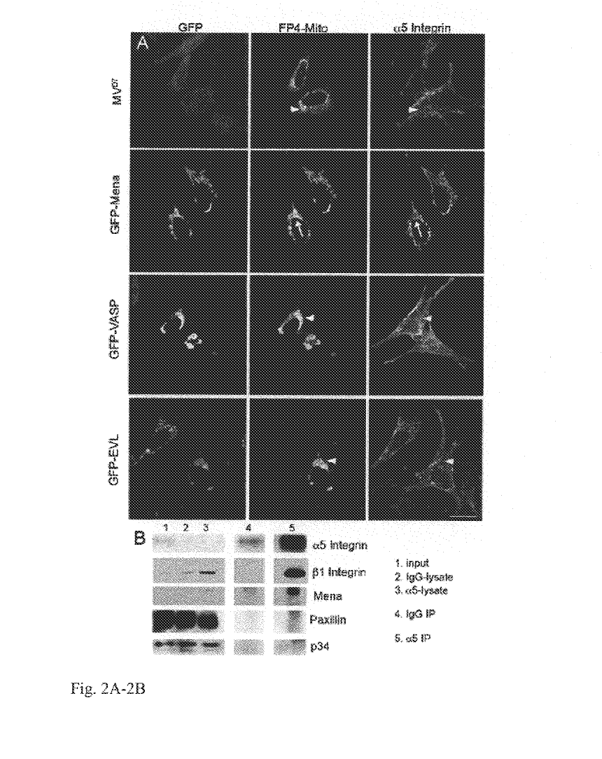

Primary meningeal fibroblasts (present in cortical neuronal preparations) transfected GFP-tagged FP4-Mito and stained with anti-Mena and anti-α5 antibodies were examined and the expected redistribution of Mena observed (not shown) along with an unanticipated recruitment of α5 integrin to the mitochondrial surface (FIG. 1A). In untransfected cells, α5 localized as expected: to the lamellipodium, to small adhesion sites behind the lamellipodium (likely FXs) and to larger structures resembling Fas (Zamir et al., 2000). Recruitment of α5 to mitochondria by FP4-Mito coincided with a loss of detectable α5 integrin signals elsewhere in the cell (FIG. 1). Expression of FP4-Mito in several cell types, including NIH3T3 cells, Rat2 cells, MBA-MDA-231 cells and NMuMg cells (data not shown) all caused re-localization of both α5 integrin and Ena/VASP proteins to mitochondria, but not of other FX/FA components such as vinculin. Expression of a control construct, “DP4-Mito”, that cannot bind Ena/VASP failed, as expected, to recruit Ena/VASP proteins to mitochondria and had no effect on α5 localization (FIG. S1 A). In MVD7 cells, a line derived from Mena/VASP double-mutant mice that express only trace levels of EVL (Bear et al., 2000), FP4-Mito expression failed to recruit α5 integrin detectably to mitochondria (FIG. 2A). To confirm these results biochemically, mitochondria were isolated from NIH3T3 cells and it was found that Mena and α5 were both enriched in the mitochondrial fraction from cells expressing FP4-Mito but not DP4-Mito. Together, these results indicate that Ena/VASP proteins mediate α5 recruitment to the mitochondrial surface by FP4-Mito. To determine if Ena/VASP could recruit other integrins or FA components to mitochondrial surfaces, cells expressing FP4-Mito were stained with antibodies to αv- and α6-integrins and vinculin and it was observed that their distributions were unaffected (FIG. 1B). FP4-Mito expression did, however, recruit a fraction of the β1 integrin pool to mitochondria, likely by association with its dimerization partner α5 integrin, while the remaining 131 integrin was likely dimerized with other α integrins, such as α6, that are not affected by FP4-Mito expression (FIG. 1B). Therefore, Ena/VASP-dependent recruitment of α5β1 to mitochondria via FP4-Mito is specific and not a general recruitment of multiple integrins or focal adhesion proteins.

The observed recruitment to mitochondria likely involves capture of α5β1-containing vesicles with their cytoplasmic tails accessible to bind the mitochondrial tethered Ena/VASP proteins directly or indirectly. Integrin trafficking is exquisitely regulated and has been studied extensively during biosynthesis, adhesion disassembly, integrin redistribution and regulation of growth factor receptor trafficking among other processes (Caswell et al., 2009; Margadant et al., 2011). Whether the putative α5β1-containing vesicles were captured by Ena/VASP during a particular stage of trafficking was investigated. FP4-Mito expressing cells were stained with antibodies that recognize vesicle populations involved in some of the known α5β1 trafficking pathways including: EEA1, an early endosomal marker, Rab7, a marker for vesicles containing activated β1 integrins (Arjonen et al., 2012) and Rab11, which decorates α5β1-containing vesicles as they pass through the perinuclear recycling compartment and is retained during integrin recycling to the plasma membrane (Margadant et al., 2011). No notable enrichment of these markers was observed on the α5β1-coated mitochondria of FP4-Mito expressing cells.

FP4-Mito recruits α5 to mitochondria through Mena. Next, it was investigated whether Mena, VASP and EVL could each recruit α5 to mitochondria in FP4-Mito expressing cells. As before, FP4-Mito expression in MVD7 cells failed to recruit α5 integrin to mitochondria (FIG. 2A). Expression of GFP-Mena, but not GFP-VASP or GFP-EVL in MVD7 cells expressing FP4-Mito resulted in α5 recruitment to mitochondria (FIG. 2A) Therefore Mena but not VASP or EVL, recruits α5 integrin to FP4-Mito-decorated mitochondria. To determine if endogenously expressed Mena and α5 integrin form complexes within cells, α5 integrin was immunoprecipitated from NIH3T3 cell lysates followed by Western blot analysis (FIG. 2B). As expected, β1 integrin was enriched in the immunoprecipitates, as was Mena, indicating these proteins are in complex in cells. To verify the specificity of the Mena-α5 co-immunoprecipitation further, α5 immunoprecipitates were analyzed using antibodies for paxillin and p34, a component of the Arp2/3 complex and found that neither protein was detectable in the α5 immunoprecipitate (FIG. 2B). Therefore, Mena is present in specific complexes with α5 integrin.

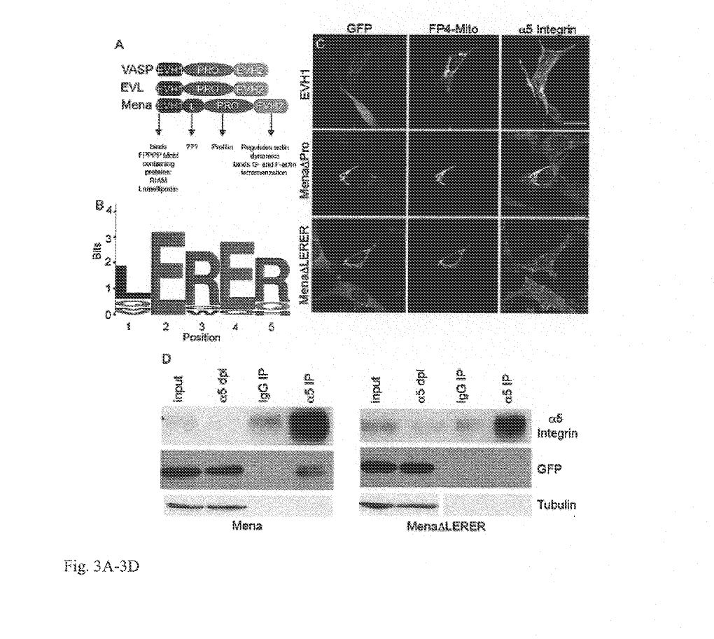

The LERER repeat mediates Mena:α5 interaction. Having determined that Mena and α5 associate within cells, next the regions in Mena required to interact with α5 integrin were mapped by transfecting FP4-Mito into cells expressing a series of previously characterized GFP-tagged Mena deletion mutants (Loureiro et al., 2002). As expected, GFP-tagged EVH1 domain of Mena was recruited to FP4-Mito labeled mitochondria, however α5 integrin localization was unaffected (FIG. 3B) indicating that additional sequences within Mena are required to interact with α5. A Mena mutant lacking the proline-rich region (MenaΔPro) co-recruited α5 integrin to mitochondria while a mutant lacking the LERER repeat (MenaΔLERER) exhibited no change in α5 distribution (FIG. 3B), indicating that the LERER repeats, but not the proline-rich central core within Mena, is required to recruit α5 to FP4-Mito-labeled mitochondria. α5 was immunoprecipitated from MVD7 cells expressing either intact GFPMena or GFP-MenaΔLERER and it was found that GFP-Mena but not GFP-MenaΔLERER, was detected in the α5 immunoprecipitates (FIG. 3C). Therefore, the LERER repeat is necessary for complex formation between Mena and α5 integrin.

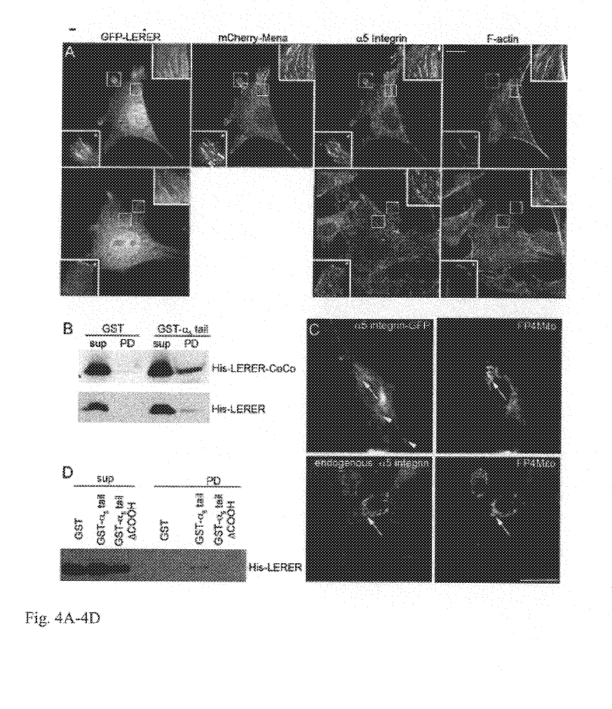

α5 integrin binds directly to the LERER repeat region. Since the LERER repeat is necessary for the Mena:α5 complex, it was investigated whether it was sufficient to mediate the interaction. When the isolated LERER repeat from Mena was expressed as a GFP fusion (GFP-LERER) in MVD7 cells expressing mCherry-Mena, GFP signal appeared enriched in peripheral FAs containing both α5 integrin and mCherry-Mena, but was weak or undetectable in adhesions containing either α5 or mCherry-Mena, but not both (FIG. 4A). When expressed in parental MVD7 cells, GFP-LERER localized predominantly to more central adhesion-like structures where it overlapped partially with α5, but was not detected within peripheral FAs (4A, lower panel). GFP-LERER was also enriched at the cell edge and present diffusely throughout the cytosol and in the nucleus regardless of whether mCherry-Mena was expressed. Therefore, the LERER repeat is sufficient to localize GFP to at least a subset of α5-positive adhesions and, when co-expressed with mCherry-Mena, the LERER repeat appears enriched in adhesions containing both α5 and Mena.

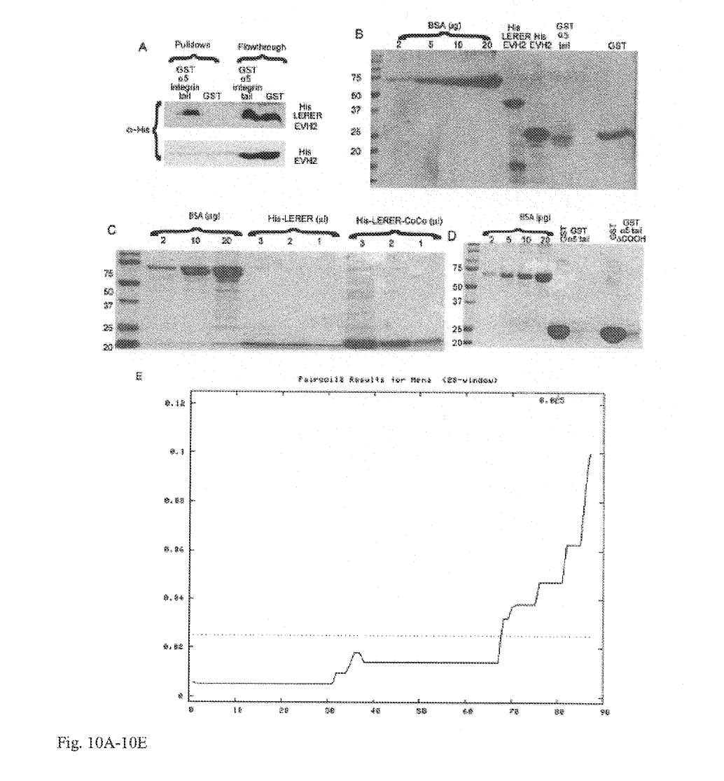

It was next asked whether the LERER repeat could bind directly to the α5 cytoplasmic tail. Purified His-tagged LERER repeat protein (His-LERER) was mixed with purified GST-α5 cytoplasmic tail (GST α5 tail) immobilized on glutathione beads (FIG. 10). After incubation, GST- and GST-α5 beads containing the His-LERER bound fraction were recovered and analyzed by western blotting along with aliquots of unbound protein from the supernatant. A small, but clearly detectable fraction of the input His-LERER was bound by GST-α5 but not GST (4B, lower panel) indicating that the LERER repeat can bind directly to the α5 tail. The His-LERER construct was then adapted to produce protein that would reflect the state of the LERER repeat within intact Mena more accurately. Analysis of the LERER repeat region sequence using paircoils2 (McDonnell et al., 2006) predicted that the first 66 residues of the LERER repeat region likely form a coiled-coil structure (p=0.0051 for residues 1-36; p=0.0139 for 37-66, FIG. 10) suggesting that it may dimerize or form higher-order multimers. The probability that the LERER repeat normally multimerizes is increased further because Mena, like all Ena/VASP proteins forms stable tetramers via a C-terminal coiled-coil within the EVH2 domains (Barzik et al., 2005; Kühnel et al., 2004). It was hypothesized that linking the LERER repeats to the tetramer-forming coiled coil within Mena would promote formation of, or stabilize, LERER multimers that would bind the α5 tail more robustly due to increased avidity. Constructs were generated to produce His-LERER fused, via a flexible 6-residue spacer, to the tetramerizing coiled-coil from Mena (His-LERER-CoCo) or fused to the entire EVH2 domain (His-LERER-EVH2). As predicted, compared to His-LERER, increased recovery of His-LERER-CoCo was observed (FIG. 4B) and His-LERER-EVH2 in the fraction bound to GST-α5 tail, while no EVH2 alone was bound detectably (FIG. 10).

Next, the sequences within the α5 tail that bind Mena were delineated. First, it was asked whether the free C-terminal end of the α5 tail was required for the interaction using an α5 construct fused to GFP tag at its C-terminus (α5-GFP). NIH3T3 cells cotransfected with α5-GFP and FP4-Mito exhibited no detectable enrichment of α5-GFP on the mitochondrial surface, while endogenous α5 (detected by immunofluorescence) was clearly recruited to FP4-Mito (FIG. 4C). Therefore, the GFP tag on the α5 tail prevented the α5 integrin-Mena interaction. Further mapping experiments using peptides derived from the 28aa α5 cytoplasmic tail sequence indicated that the C-terminal-most portion of the tail could bind His-LERER (data not shown). When a GST-α5 cytoplasmic tail lacking the C-terminal five residues (GST-α5 tail ΔCOOH) was used in the binding assay, His-LERER was not detectable in the bound fraction (FIG. 4D). These results indicate that the LERER repeat of Mena can bind directly to the α5 through an interaction requiring the C-terminal portion of α5.

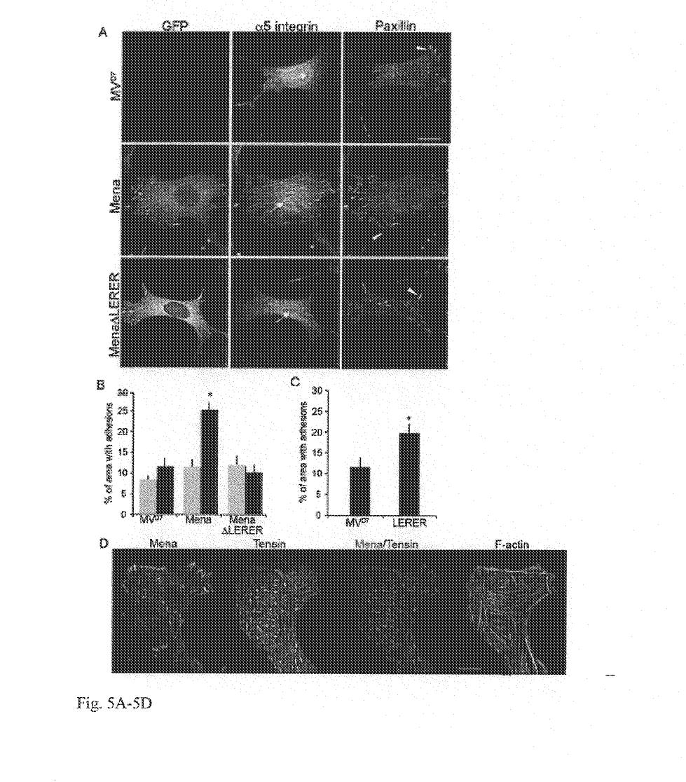

The Mena LERER repeat region modulates the subcellular distribution of α5. Like all components of cell:matrix adhesions, Mena and α5β1 levels vary dynamically within these structures as they mature during cell spreading and migration (Zaidel-Bar et al., 2003). Whether the Mena:α5 interaction influences the distribution of either molecule to the different types of adhesive structures was studied. In fibroblasts cultured on FN, α5β1 is found typically in nascent FXs, FAs and FBs. MVD7 cells expressing GFP-Mena exhibited extensive co-localization of Mena, α5 and paxillin in peripheral FAs, while the cell center displayed robust α5 signal typical of FBs that contained little, if any detectable GFP-Mena (FIG. 5A). To examine FBs directly, endogenous Mena was localized by immunofluorescence in NIH3T3 fibroblasts transiently transfected with GFP-tensin, a major component of FBs, (Zamir et al., 2000) and only very weak overlap of Mena with tensin in central FBs was found (FIG. 5D). Interestingly, parental MVD7 cells contained peripheral FAs with α5 and paxillin, but lacked any prominent FB-like α5 signal. Similarly, MVD7 cells expressing GFP-MenaΔLERER contained α5, paxillin and GFP-MenaΔLERER within peripheral FAs but lacked α5-positive FBs in the cell center. Expression of GFP-VASP also failed to restore α5-positive FB-like adhesions in MVD7 cells. The fraction of the ventral cell surface containing α5 or paxillin was similar in MVD7 and GFP-MenaΔLERER cells, while cells expressing GFP-Mena had approximately double the area of α5-positive adhesions relative to paxillin (FIG. 5B). FACS analyses with anti-α5 antibodies indicated that similar levels of α5 were present on the surface of parental MVD7 cells, MVD7 cells expressing GFP-MenaΔLERER and MVD7 cells expressing GFP-Mena. Additionally, ELISA measurements of biotinylated α5 integrin from adherent cells revealed no significant differences in surface levels of α5 across these different cell lines (data not shown). These data indicate that altered distribution of α5 was likely not a consequence of defects in trafficking to, or maintenance of α5 at the cell surface. Therefore, the LERER repeat is necessary for Mena-dependent formation or maintainance of α5-positive central FBs, normally a large fraction of the total area containing α5-positive adhesions. Interestingly, expression of GFP-LERER alone was sufficient to increase the total area of α5-containing adhesions in MVD7 cells (FIGS. 5C; 4A).

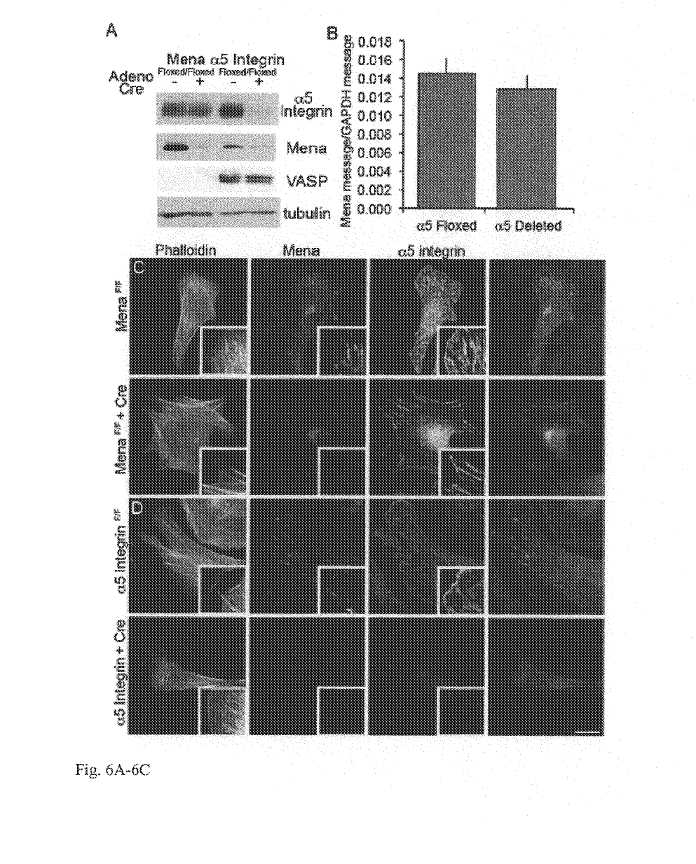

To confirm these results in a second cell type, primary fibroblasts were isolated from perinatal mice homozygous for a conditional Mena allele (Mena^{Floxed}) to examine formation of α5-containing FBs after Mena deletion in culture. To excise the Mena^{Floxed} allele, cells were infected with adenovirus expressing either GFP-Cre recombinase or GFP alone (FIGS. 6A,C). In GFP-infected control fibroblasts, Mena and α5 co-localized at the leading edge and in peripheral FAs, while α5, but not Mena, was also present in central FBs (FIG. 6C). In Mena-deficient cells, α5 localized to the leading edge and peripheral FAs but was not detected in central FB-like adhesions (FIG. 6C). Therefore, absence of Mena in either primary fibroblasts or MVD7 cells results in loss of central FBlike α5 adhesions.

The effects of α5 deletion on Mena were tested. Primary fibroblasts isolated from perinatal mice homozygous for an α5floxed allele (van der Flier et al., 2010) were infected with Cre-expressing or control adenovirus (FIGS. 6 A, D). Surprisingly, reduction in α5 levels resulted in a concomitant loss of Mena. Interestingly, VASP levels were unaffected by α5 deletion indicating that the effect was specific to Mena and not all Ena/VASP proteins. To determine whether the α5-dependent loss of Mena involved changes its mRNA, Cre-treated and control fibroblasts were analyzed by qRT-PCR for Mena mRNA and it was found that the message levels were unaffected (FIG. 6B). Therefore, elimination of α5 in primary fibroblasts induces a post-transcriptional reduction in Mena protein levels.

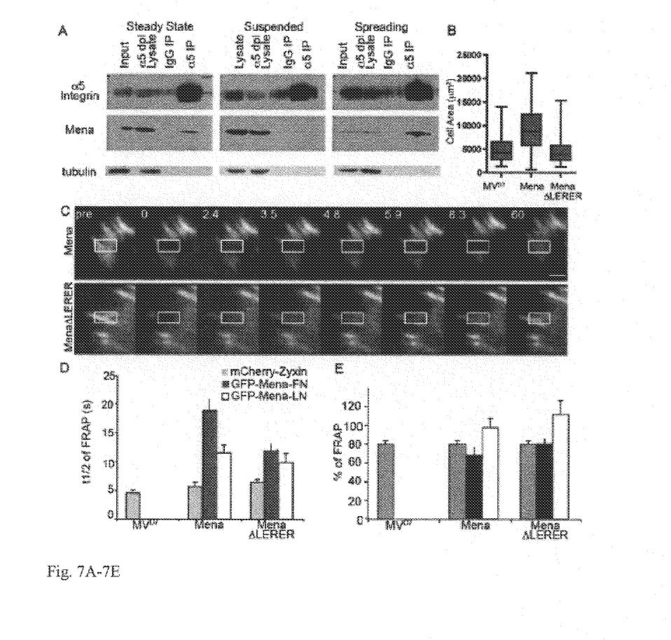

Adhesion to FN increases the amount Mena in complex with α5. The activation state of integrins often modulates interactions with their cytosolic binding partners. To determine whether the Mena:α5 interaction is sensitive to α5β1 activation, α5 complexes were immunoprecipitated from adherent, suspended and spreading cells. Interestingly, compared to adherent cells in steady-state conditions, significantly more Mena was detected in complex with α5 30 min after plating cells on FN (FIG. 7A). In contrast, the amount of Mena in complex with α5 was reduced in suspended cells. To determine whether the observed increase in Mena:α5 association had functional consequences, the cell area of MVD7, or MVD7+GFP-Mena or MVD7+GFPMenaΔLERER was measured 30 minutes after plating on FN-coated coverslips (FIG. 7B).

MVD7 cells expressing GFP-Mena were significantly more spread (p<0.01 ANOVA LSD) compared to both MVD7 cells and MVD7+GFP-MenaΔLERER cells, which spread equivalently. Therefore, the increased amount of α5:Mena in complex during cell spreading correlates with increased cell spreading. Fibroblasts spread on FN in distinct steps initiated as integrins bind to FN and trigger rapid actin-polymerization-driven, adhesion-independent membrane extension followed by a distinct phase during which adhesions form dynamically, providing traction required for further spreading (Zhang et al., 2008). As fibroblasts attach to, and spread on FN, Mena localizes to the leading edge and to nascent β1-positive peripheral adhesions as they appear (Zhang et al., 2008).

Whether the adhesion dependent increase in Mena interaction with α5 affects its stability in FAs during spreading was investigated also. FRAP (Fluorescence Recovery After Photobleaching) analysis was used to measure the recovery dynamics after photobleaching of GFP-Mena or GFP-MenaΔLERER in nascent, peripheral adhesions within cells plated for 30 minutes on FN (FIG. 7C-E). The t_{1/2} of fluorescence recovery was significantly greater for GFP-Mena than GFP-MenaΔLERER (18.9+/−1.4 s vs.11.9+/−1.6 s, p<0.01), but the overall percentage of FRAP was unchanged (FIG. 7E). In contrast, the t_{1/2} of FRAP of the FA component zyxin, did not vary among MVD7 parental cells, cells expressing GFP-Mena or GFP-MenaΔLERER (FIG. 7D). Since the t_{1/2} of FRAP of zyxin, which binds Mena directly (Drees et al., 2000) and helps localize it to FAs (Hoffman et al., 2006), was unaffected by GFP-MenaΔLERER, we conclude that expression of this mutant did not induce a general perturbation of FA protein dynamics. Interestingly, the t_{1/2} of FRAP of Mena and MenaΔLERER was equivalent 24 hours after plating on FN (data not shown). When plated for 30 min on laminin (LN), an ECM protein bound by a distinct set of integrins, the dynamics of both Mena and MenaΔLERER were equivalent to those observed for MenaΔLERER in cells plated 30 minutes on FN. Taken together, these data indicate that FN binding by α5β1 during cell spreading reduces the turnover of Mena, dependent upon its LERER repeat, which mediates direct binding to α5.

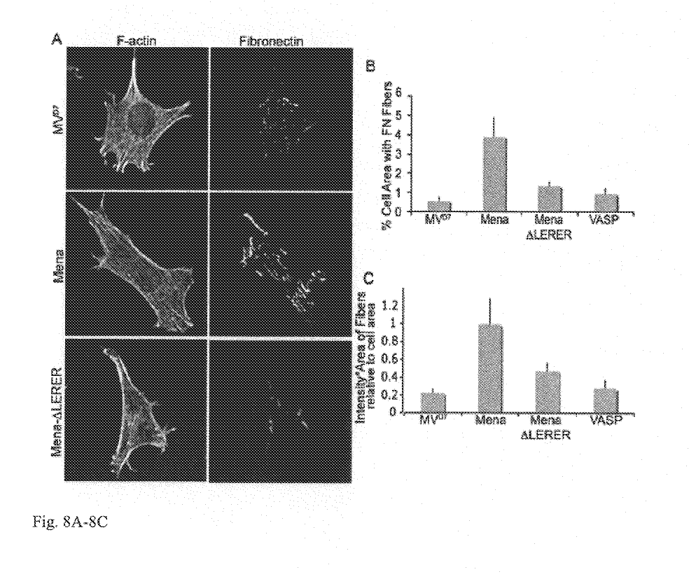

The Mena:α5 interaction is required for normal FN fibrillogenesis. α5β1 remains attached to FN as it moves centripetally along stress fibers towards the cell center, forming FBs and generating the tension required to initiate fibrillogenesis (Danen et al., 2002; Pankov et al., 2000). The absence of central α5β1-positive FBs in MVD7 and MenaΔLERER cells (FIG. 5) led us to ask whether Mena:α5 binding is required for α5β1-dependent FN fibrillogenesis. Parental MVD7 cells and MVD7 cells expressing GFP-Mena, GFP-MenaΔLERER, or GFP-VASP were plated overnight on vitronectin-coated coverslips. Four hours after adding FN to the media, cells were fixed and stained to identify FN fibrils (FIG. 8). MVD7+GFP-Mena cells generated typical FN fibrils that aligned with stress fibers and FBs, while parental MVD7 cells, and MVD7 cells expressing either GFP-MenaΔLERER or GFP-VASP formed significantly less fibrillar FN (p<0.05, ANOVA) (FIG. 8)

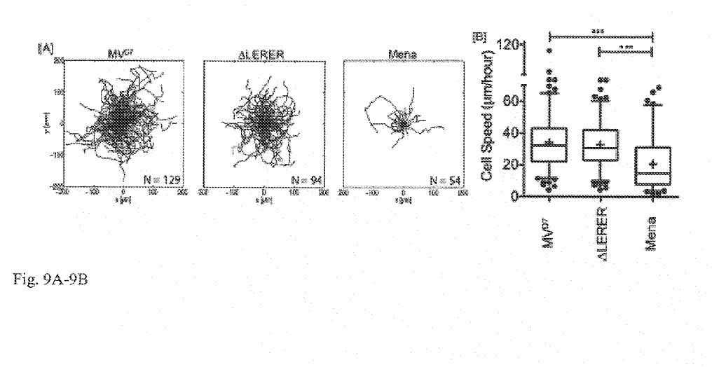

The Mena:α5 interaction influences cell motility. Both Mena and α5β1 exert context-dependent effects on cell motility, prompting investigation of whether disrupting their interaction would affect cell migration. MVD7 cells exhibit a hypermotile phenotype, migrating roughly twice as fast as MVD7 cells expressing GFPMena at levels typical for fibroblasts (Bear et al., 2000). Time-lapse movies of MVD7 cells and derivative lines expressing GFP-Mena and GFP-MenaΔLERER were analyzed to determine cell speed and directional persistence (FIG. 9). Directional persistence of MVD7 cells was unaffected by expression of Mena or MenaΔLERER (not shown). As expected, MVD7 cells migrated about twice as fast as cells expressing GFP-Mena, however, MVD7 cells expressing GFP-MenaΔLERER moved at a rate similar to MVD7 cells (FIG. 9B) indicating that α5 binding might be required for Mena to modulate MVD7 cell motility.

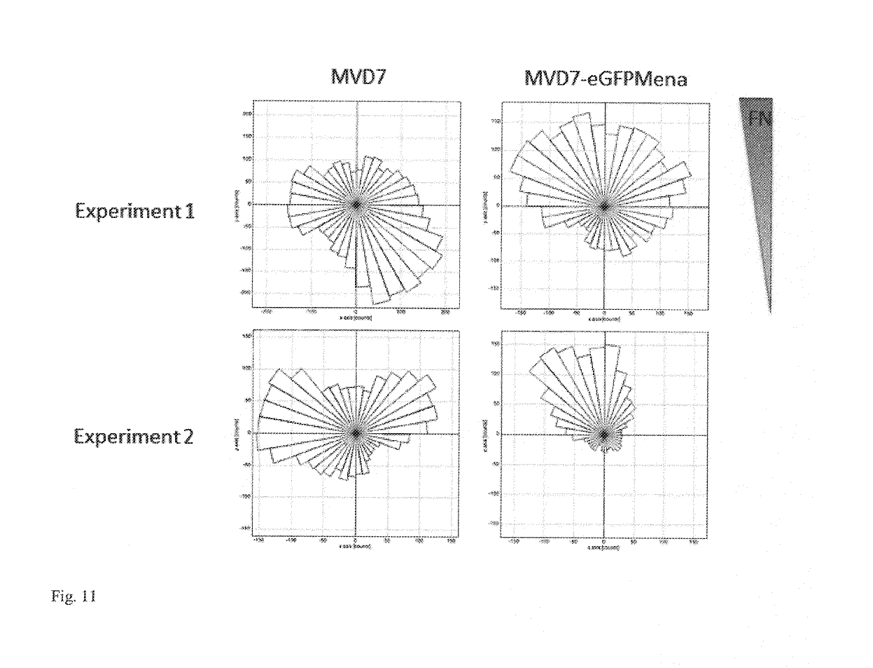

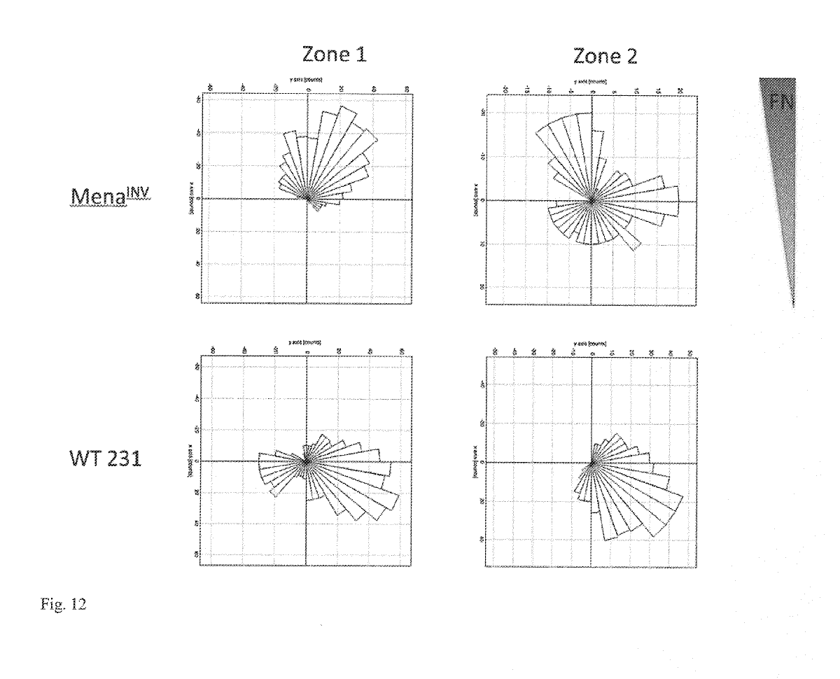

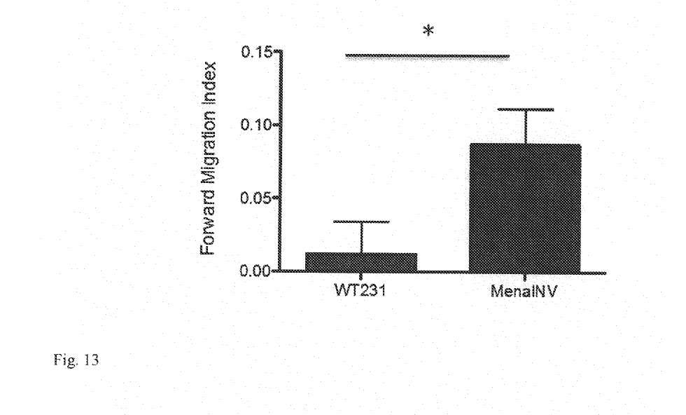

FIGS. 11-13 show fibroblasts that do not express Mena no longer respond to a fibronectin gradient. However, when Mena^{INV} is present, the cells migrate towards higher concentrations of fibronectin. This is further data demonstrating the importance of Mena (Mena^{INV}) in cell migration.

### Discussion

Cell motility and morphogenesis are dynamic, highly regulated processes that require continual remodeling of the cytoskeleton as well as cell:cell and cell:matrix adhesions. Requirements for Ena/VASP in all of these processes have been demonstrated in a wide range of systems. While Ena/VASP influences the formation, morphology and dynamics of cellular protrusions by regulating actin polymerization through a mechanism that is now coming into focus (Bear and Gertler, 2009; Hansen and Mullins, 2010), exactly how Ena/VASP affects adhesion is not well understood. This study identifies a direct connection between Mena and α5 integrin required during cell spreading and migration on FN and for FB formation and proper FN fibrillogenesis. Along with promoting α5β1 function inside-out, the Mena:α5 interaction is enhanced outside-in by FN binding to α5β1.

When validating the findings in primary fibroblasts isolated from α5^{FLOXED} or Mena^{FLOXED} animals, it was found that acute depletion of Mena protein caused a loss of central α5-containing FB-like adhesions. Interestingly, acute α5 depletion resulted in loss of Mena protein without affecting Mena mRNA levels. Therefore, in primary fibroblasts that normally express both α5 and Mena, loss of α5 causes a reduction in Mena levels either by blocking Mena translation or inducing its degradation. Consistent with this idea, integrins and FA proteins form complexes with the mRNA translation machinery (de Hoog et al., 2004; Humphries et al., 2009), and adhesion to FN triggers α5β1-dependent translation (Gorrini et al., 2005; Chung and Kim, 2008). FA proteins are also regulated by proteolytic enzymes (Franco and Huttenlocher, 2005) and by ubiquitinmediated proteosome degradation (Huang et al., 2009). Interestingly, however, Mena and α5 are each normally expressed in cells that lack the other, for example cultured cortical neurons contain Mena but lack detectable α5 (Gupton and Gertler, unpublished). Therefore, cells that normally express both proteins must have specific regulatory mechanisms that coordinate Mena levels with α5.

This direct, specific Mena:α5 interaction requires the C-terminal 5 of the 28 residue α5 cytoplasmic tail and is blocked by tagging the tail at its C-terminus Interestingly the tight junction protein ZO1 was recently identified as an α5 interacting protein (Tuomi et al., 2009), and binds residues next to those required for Mena binding. ZO1 interactions help target α5β1 to the lamellae of lung cancer cells. Whether Mena and ZO1 bind to α5 simultaneously is unknown, but it is interesting that complexes containing both VASP and ZO1 have been reported (Comerford et al., 2002), suggesting that Ena/VASP:ZO1 interactions may have specific functions. Mena binding to α5 requires the LERER repeat, a region spanning 91 or 121 amino acids with 13 or 15 repeats of the 5-residue LERER motif in mouse and human, respectively. Whether each repeat can bind an α5 tail is unknown, however, it is possible that multiple α5 tails could bind LERER repeats within each subunit of a Mena tetramer, raising the interesting possibility that Mena clusters α5β1, thereby strengthening FN binding by increased avidity. Mena promotes actin polymerization in cell protrusions (Bear and Gertler, 2009), within FAs and in sarcomeric units along Factin bundles attached to FAs of endothelial cells (Furman et al., 2007). The contractile forces exerted by endothelial cells and myosin light chain phosphorylation levels are proportional to the total level of Ena/VASP function (Furman et al., 2007) and VASP regulates smooth muscle cell contractility (Defawe et al., 2010). Therefore, Mena might contribute to contractile forces that generate conformational changes that permit highaffinity catch bonds between α5β1 and FN (Friedland et al., 2009; Kong et al., 2009). In this model, cells incapable of forming Mena:α5 complexes would remodel FN less efficiently because of reduced ability to generate forces needed to expose sites buried within FN, including the synergy site which enhances α5β1 binding and the self-association sites that dimerize FN (Schwarzbauer and DeSimone, 2011).

Despite its role in fibrillogenesis, Mena is barely detectable in FBs compared to FAs, as are two other molecules important for fibrillogenesis: FAK (Hie et al., 2004) and ILK (Vouret-Craviari et al., 2004; Stanchi et al., 2009; Zamir et al., 2000). Mena may cluster α5β1 and strengthen FN binding within FAs before α5β1:FN complexes begin moving towards central FBs. Alternatively, Mena:α5 interactions could target FAs for maturation by altering α5 dynamics and stability within FAs. Consistent with this possibility, deletion of the LERER repeat increased turnover of Mena in nascent adhesions formed during cell spreading.

The inability of α5-GFP to bind Mena may perturb α5 function in some contexts. The original description of α5-GFP included a comprehensive, convincing set of controls demonstrating that α5-GFP functioned equivalently to untagged α5 in migration and spreading when expressed in α5-deficient CHO B2 cells (Laukaitis et al., 2001). Some CHO cell lines (Benz et al., 2009) including CHO B2 lack detectable Mena protein (Riquelme and Gertler, unpublished), therefore perturbation of Mena-dependent α5 function by GFP tagging would not be expected. The consequences of disrupting the Mena:α5 interaction by GFP-tagging will likely be cell-type- and context-dependent. Along with the potential limitations of α5-GFP, we found that use of the FP4-Mito system to block Ena/VASP function can also block α5 function, an effect that must be considered when using this tool in α5-expressing cells. Our lab and others have used FP4-Mito to study Ena/VASP function in a variety of systems and thus far, most of the conclusions from these studies have been validated by experiments conducted in MVD7 cells (Loureiro et al., 2002; Bear et al., 2002), primary neurons isolated from Mena/VASP/EVL triple-null embryos (Dent et al., 2007) or Ena mutant Drosophila (which lack both α5 and the LERER-repeat) (Gates et al., 2007). The Peifer lab, however, has demonstrated that FP4-Mito expression in flies causes a partial co-depletion of Dia through association with Ena, raising the possibility that it could induce phenotypic effects that may be more severe than the Ena null state (Homem and Peifer, 2009). The LERER repeat is not found in VASP, EVL or the invertebrate and Dictyostelium Ena/VASP orthologs. Interestingly, fibronectin, α5β1 and the Mena LERER repeat are all vertebrate-specific adaptations (Whittaker et al., 2006), raising the possibility that they co-evolved. The Mena:α5 interaction is highly regulated: loss of adhesion reduces the interaction while acute FN binding during cell spreading increases both levels of the complex and the residence time of Mena within FAs. Interestingly, though VASP is not known to bind any integrin subunit directly, it promotes inside-out activation of β1- and β2-containing integrins indirectly through adaptor or signaling intermediates (Deevi et al., 2010). VASP functions in cross-regulation between αVβ3 and α5β1 (Worth et al., 2010): loss of P3 function reduces phosphorylation of a PKAdependent site within VASP near its EVH1 domain, allowing it to bind FPPPP-repeats within RIAM, an adaptor that mediates Rap-GTPase-driven integrin activation (Lafuente et al., 2004). The VASP:RIAM complex associates with the β subunit-binding protein talin (Anthis and Campbell, 2011) causing α5β1 activation at peripheral adhesions (Worth et al., 2010). Others, however, find that RIAM can promote integrin activation by talin independently of Ena/VASP (Lafuente et al., 2004; Lee et al., 2009). The Mena EVH1 domain binds many of the same ligands as VASP (Ball et al., 2002) connecting it to integrins through RIAM or other FA proteins containing EVH1-binding sites, such as vinculin and zyxin, that associate with β subunits indirectly. Juxtaposition of its EVH1 domain and LERER repeat may enable Mena to connect directly to α5 and indirectly to β1 simultaneously. In addition, Ena/VASP proteins can form mixed tetramers (Ahern-Djamali et al., 1998) that could combine Mena:α5 binding with VASP- or EVL-specific properties while diluting potential LERER-repeat clustering of α5β1. Here we found that rescue of the MVD7 hypermotile phenotype by GFP-Mena required the LERER repeat; however, previously we found that GFP-Mena and GFP-MenaΔLERER rescued the MVD7 hypermotility phenotype equivalently as did GFPVASP or GFP-EVL (Loureiro et al., 2002). That GFP-MenaΔLERER was expressed stably and exhibited subcellular distribution similar to GFP-Mena, as previously observed (Loureiro et al., 2002), was verified. The divergent results may have arisen from differences in methods and reagents used in the 10-year old study that cannot be tested, including FN or other reagents, or use of cells adapted to CO₂-independent media as opposed to the current enclosed environmental chamber used for live-cell imaging. In addition, the current sample size is much larger: 372 MVD7 cells expressing GFP-MenaΔLERER from 4 separate 12-hour time-lapse movies were analyzed compared to 22 cells from 2 separate 4-hour experiments in the older study.

Why is the LERER repeat required for Mena to rescue MVD7 cell spreading and motility? Ena/VASP deficiency reduces cellular capacity to generate actin-driven protrusive forces that drive lamellipodial and filopodial extension and propulsion of the intracellular pathogen Listeria monocytogenes, even though the actin networks formed during these process are organized differently. In general, expression of Mena, VASP or EVL each rescue the actin polymerization-dependent phenotypes evident in the absence of Ena/VASP in MVD7 cells or in primary neurons from triple Mena/VASP/EVL null embryos (Loureiro et al., 2002; Geese et al., 2002; Applewhite et al., 2007; Dent et al., 2007). GFP-Mena expression in MVD7 cells produces rapidly extending, but shortlived lamellipodia that cannot contribute efficiently to locomotion (Bear et al., 2002).

Conversely, lamellipodia in parental MVD7 cells protrude slowly but are more stable and can contribute to productive translocation likely by adhering to the substratum before the protrusion cycle ends (Bear et al., 2002). These differences probably arise from changes in the actin network: high Ena/VASP activity produces longer, sparsely branched filament networks that, absent stabilizing interconnections, become increasingly prone to buckle against the membrane as they elongate due to their inherent flexibility (Mogilner and Oster, 2003). Importantly, the net effect of Ena/VASP on actin polymerization leads to context-dependent morphological outputs contingent on variables including location, density, and strength of adhesion sites along with the relative amounts of actin bundling and crosslinking proteins (Mogilner and Keren, 2009). By coupling its stimulatory effect on barbed end elongation with the ability to bind and cluster α5β1, Mena could present activated but unbound integrins right at the tips of lamellipodia and filopodia consistent with the proposed “sticky fingers” mechanism for haptotaxis (Galbraith et al., 2007). In addition, through its role in FN remodeling, Mena may help form the interstitial fibrillar network that serves both as a migration substrate and template that organizes growth factors and other ECM components into spatially organized cues that elicit complex, coordinated responses (Hynes and Naba, 2012) when touched by the sticky fingers of cells in transit. Over the past several years, new evidence has implicated both α5β1 (Muller et al., 2009; Caswell et al., 2008; Valastyan et al., 2009) and Mena (Robinson et al., 2009; Philippar et al., 2008; Roussos et al., 2010) in breast cancer invasion and metastasis through effects on EGFR (Gertler and Condeelis, 2011). Many carcinomas types exhibit elevated levels of Mena that are, at least in breast cancer, critical for metastatic progression (Gertler and Condeelis, 2011; Roussos et al., 2010) and could involve interaction with α5. In breast cancer patients, risk of distant metastasis correlates with density of a tripartite microanatomical structure called TMEM composed of a carcinoma cell expressing Mena, a macrophage and a blood vessel all contacting each other (Robinson et al., 2009). During breast cancer progression, changes in alternative splicing produce additional Mena protein isoforms co-expressed with the canonical isoform. Primary tumor cells express Mena11a, normally an epithelial-specific isoform lost when cells undergo epithelial to mesenchymal transition (Shapiro et al., 2011; Warzecha et al., 2009). Invasive tumors stop expressing Mena11a, while a subpopulation of highly invasive, motile and chemotactic tumor cells express an invasion-specific Mena isoform, Mena^{INV} (Goswami et al., 2009). Mena^{INV} has been detected in breast cancer patients with invasive ductal carcinomas at levels proportionate to their TMEM density (Roussos et al., 2011b). Interestingly, the exon encoding the 19 amino acid INV sequence is inserted between the EVH1 domain and the LERER repeat region.

Both Mena^{INV} and α5β1 modulate EGFR function. Mena^{INV} sensitizes tumor cells to EGF, allowing invasive or chemotactic responses to 25-50 fold lower EGF concentrations than in cells lacking this isoform, and leads to substantially increased metastatic burden (Philippar et al., 2008; Roussos et al., 2011a). Upon inhibition of αVβ3 or in cells expressing the mutant form of the p53 tumor suppressor, α5β1 forms complexes with EGFR through their mutual cytosolic binding partner, RCP (Caswell et al., 2008; Muller et al., 2009). Association of α5β1-RCP with EGFR leads to coordinated recycling that targets α5β1 and EGFR to the front of cells and promotes 3D invasion. Complex formation between α5β1 and EGFR also dysregulates signaling downstream of both receptors. Interestingly, a recent report demonstrated that increased expression of Mena and mutant p53 were highly correlated in patients with infiltrating ductal carcinomas (Toyoda et al., 2011).

### Materials and Methods

Western Blotting/Immunoprecipitation. Standard procedures were used for protein electrophoresis, western blotting and immunoprecipitations. Western blots were developed using HRP secondary antibodies and ECL reagent (Amersham). For α5 integrin immunoprecipitation, cells were lysed at 4° C. in CSK buffer (Humphries et al., 2009) with intermittent agitation for 20 minutes, passed through a 23.5 gauge needle, and the supernatant was kept after spinning 15 minutes at 21,000×g. Lysates were precleared with protein A beads for two hours, incubated with an α5 integrin antibody (Millipore, 1928) for two hours at 4° C., and then captured with BSA blocked protein A beads for two hours. Beads were washed three times in lysis buffer, and proteins were eluted in sample buffer. Western blots were probed for α5 integrin (Santa Cruz sc-166681) Mena (Lebrand et al., 2004), Paxillin (Signal Transduction laboratories), p34 (Millipore, 07-227), β1 integrin (Millipore, 1949), GFP (Clontech, JL-8), GAPDH(Signal trandsduction laboratories, 2118), porin (Molecular Probes, A-21317), tubulin (DM1A), His tag (Sigma, H1029), and VASP polyclonal (Lanier et al., 1999). The function blocking α5 integrin antibody BIIG2 was purchased from Iowa University Developmental Studies Hybridoma bank, and used at 20 μg/ml.

Mitochondrial Purification: Mitochondria were isolated from NIH3T3 cells expressing either FP4-Mito or DP4-Mito using paramagnetic beads conjugated to an antibody specific for mitochondrial protein Tom34 (Miltenyi Biotec, according to manufacturer instructions).

Binding Assays: GST-α5 integrin constructs and His-tagged variants of the LERER repeat region were expressed and purified from E. Coli. 10 nM α5 integrin cytoplasmic tail was immobilized on Glutathione beads and incubated for 1 hour, 4° C. with 200 nM His-LERER variants at constant agitation in PBS with 0.1% TritonX-100 and 2 mM βME. Beads were washed three times, and proteins were eluted in sample buffer, and assayed by western blot.

Microscopy—Cells were fixed in 4% paraformaldehyde in PHEM buffer warmed to 37° C. for 20 minutes. Cells were permeabilized in 0.2% TX-100 and blocked in 10% Donkey Serum. Primary antibodies used for immunofluorescence include α5 integrin (Millipore 1928), integrin α4 [PS/2] (Abcam ab25247), integrin αv [RMV-7] (Abcam ab63490), integrin α6 [GoH3] (ab105669), vinculin (Sigma), Mena, GFP (Clontech, JL-8), paxillin (BD Transduction, 610052), Rab7 (Cell Signaling, 9367S), Rab11 (Cell Signaling 5589), and EEA1 (Cell Signaling, 3288S). F-actin was stained with AlexaFluor Phalloidin (Invitrogen). Z series of images were taken on an Olympus microscope with a 60× plan apo objective. Images were deconvolved using Deltavision Softworx software. FRAP was performed on a Olympus microscope using DeltaVision software and solid state 405 laser in TIRF mode with a depth of 100 nm. Images were acquired pre and post bleach with 488 and 561 solid state laser with 63×1.4 NA Plan Apochromatic objective lens (Olympus). A pre-bleach series of ten images was collected at 10 s interval, the area of interest was bleached with 50% laser power. The acquisition settings were returned to pre-bleach settings, and images were taken at adaptive time frame. Total elapsed time between the end of the pre-bleach series and the beginning of the post-bleach series was 40-90 s (median 50 s).

Sequence analysis—The murine Mena (ENAH) from UniProt (mouse: Q03173) to identify the repeat region as residues 175-252. By visual inspection, these sequence regions were divided into chunks fitting one of several motifs: a five amino acid motif roughly consistent with the form “L/M/Q-E-R/Q-E-R/Q” (SEQ ID NO:1), a seven amino acid motif roughly consistent with the 5-mer motif with the last two amino acids of the motif repeated (SEQ ID NO:2), and an eight amino acid motif roughly consistent with the 5-mer motif preceded by a repetition of the first three amino acids of the motif (SEQ ID NO:3). All sequence in the region of interest fell into one of these three motifs, with no sequence unused. A motif logo was generated for each species using each instance of the 5-mer motif, the first five amino acids of the 7-mer motif, and the last five amino acids of the 8-mer motif using the program WebLogo (http://weblogo.berkeley.edu/)

Image analysis—Cell masks of cell area were made by threshholding phalloidin images. Subsequently, threshholding was done to evenly include adhesive structures between cells within these masks, and intensity and area of these regions were measured. For analysis of photobleaching data, images were first corrected for overall photobleaching, and the integrated fluorescence intensity (Fr) inside a region that was smaller than the original bleached region by 4 pixels in x and y in each image was measured in the pre-bleach and recovery image series. Calculation of the t1/2 of recovery and percent fluorescence recovery was performed as described (Bulinski et al., 2001).

Cell Culture and Plasmids. Coverslips were coated with 10 μg/ml bovine FN (Sigma) for 2 hours at 37° C. Primary meningeal fibroblasts were cultured with cortical neurons, isolated from embryonic day 14.5 mice as described (Dent et al., 2007). Perinatal fibroblasts were isolated from postnatal day 1 mice that harbored either floxed α5 integrin (van der Flier et al., 2010) or floxed Mena (will be described in a separate publication). NIH3T3 cells, Rat2 cells, and perinatal fibroblasts were cultured in DME supplemented with 10% fetal bovine serum. Parental MVD7 cells and MVD7 cells expressing GFP-tagged Mena and Mena mutants were cultured as described (Bear et al., 2000). Mcherry FP4-Mito, GFP-LERER, and GFP-α5 integrin were introduced into MVD7 cells using Lonza nucleofection per the manufacturer protocol. pMVSCV-GFPLERER, pMVSCV-GFP-MenaΔLERER, pGEX-GST-α5 cytoplasmic tail, pGEX-GST α5 cytoplasmic tail ACOOH, pQE80L-His-LERER, pQE80L-His-LERER-CoCo, pQE80LHis-LERER-EVH2, and pQE80L-His-EVH2 were cloned using standard cloning procedures. mCherry-FP4-Mito was previously described (Bear et al., 2000). GFPtensin was a kind gift from Ken Yamada and was introduced into Rat2 cells with Lipofectamine 2000 (Invitrogen) following manufacturer's directions. GFP-α5 integrin (Laukaitis et al., 2001) was purchased from Addgene.

FN Fibrillogenesis—FN-depleted medium was prepared as described (Pankov and Momchilova, 2009). FN was fluorescently labeled with 549—NHS ester from Thermo-Scientific (46407), as directed by the manufacturer. MVD7 cells were seeded on coverslips coated with vitronectin (10 μg/ml) from Sigma (V9881) and allowed to adhere overnight. Medium was replaced with FN-depleted growth medium containing 10 μg/ml fluorescently labeled FN and incubated at 32° C. for four hours. Cells were then fixed and stained as indicated above.

Motility analysis—MVD7 cells were stained with 1 μM CMFDA (Invitrogen) and seeded overnight in growth medium at 2000 cells/cm2 on FN (10 μg/mL) coated coverglass. Media was replenished directly before imaging to facilitate addition of 10 ρg/mL of α5 blocking antibody [BD Pharmingen, 5H10-27 (MFRS)] where applicable. Two-dimensional migration was quantified by recording cell centroid displacement after live-cell imaging for 12 hrs (1 image/10 min) using a Zeiss Axiovert inverted microscope equipped with automatic stage positioning, a 5% CO₂-37° C. environmental chamber, fluorescent light source, and 10× plan-fluor objective. Resulting images were semi-automatically tracked using Imaris software (Bitplane, Inc). A custom Matlab (Mathworks) script was used to calculate migration parameters and create wind-rose plots. Cell speed is reported for the final 6 hours of the experiment to ensure steady-state. α5 integrin surface levels For assessment of α5 integrin surface levels, MVD7 fibroblasts were incubated on ice in 1% BSA, 2 mM EDTA in PBS with biotinylated α5 integrin antibody (BD Pharmingen, 557446) or biotinylated rat IgG (Jackson ImmunoResearch, 012-060-003) for 30 mins. Cells were washed, and incubated for 30 mins on ice with APC streptavidin (BD Pharm 554067) and propidium iodide. Cells were washed, resuspended and directly analyzed on a FACSCalibur (BD Biosciences). Biotinylation and analysis of surface levels of α5 integrin was performed as described (Caswell et al., 2008).

### REFERENCES

Ahern-Djamali, S. M., A. R. Corner, C. Bachmann, A. S. Kastenmeier, S. K. Reddy, M. C. Beckerle, U. Walter, and F. M. Hoffmann. 1998. Mutations in Drosophila enabled and rescue by human vasodilator-stimulated phosphoprotein (VASP) indicate important functional roles for Ena/VASP homology domain 1 (EVH1) and EVH2 domains. Molecular Biology of the Cell. 9:2157-2171.
Anthis, N. J., and I. D. Campbell. 2011. The tail of integrin activation. Trends in Biochemical Sciences. 36:191-198.
Applewhite, D. A., M. Barzik, S.-I. Kojima, T. M. Svitkina, F. B. Gertler, and G. G. Borisy. 2007. Ena/VASP proteins have an anti-capping independent function in filopodia formation. Molecular Biology of the Cell. 18:2579-2591.
Arjonen, A., J. Alanko, S. Veltel, and J. Ivaska. 2012. Distinct recycling of active and inactive β1 integrins. Traffic.
Aszodi, A., A. Pfeifer, M. Ahmad, M. Glauner, X. H. Zhou, L. Ny, K. E. Andersson, B. Kehrel, S. Offermanns, and R. Fassler. 1999. The vasodilator-stimulated phosphoprotein (VASP) is involved in cGMP- and cAMP-mediated inhibition of agonistinduced platelet aggregation, but is dispensable for smooth muscle function. EMBO J. 18:37-48. Ball, L. J., T. Jarchau, H. Oschkinat, and U. Walter. 2002. EVH1 domains: structure, function and interactions. FEBS Lett. 513:45-52.
Barzik, M., T. I. Kotova, H. N. Higgs, L. Hazelwood, D. Hanein, F. B. Gertler, and D. A. Schafer. 2005. Ena/VASP proteins enhance actin polymerization in the presence of barbed end capping proteins. J Biol. Chem. 280:28653-28662.
Bear, J. E., and F. B. Gertler. 2009. Ena/VASP: towards resolving a pointed controversy at the barbed end. J Cell Sci. 122:1947-1953.
Bear, J. E., J. J. Loureiro, I. Libova, R. Fassler, J. Wehland, and F. B. Gertler. 2000. Negative regulation of fibroblast motility by Ena/VASP proteins. Cell. 101:717-728.
Bear, J. E., T. M. Svitkina, M. Krause, D. A. Schafer, J. J. Loureiro, G. A. Strasser, I. V. Maly, O. Y. Chaga, J. A. Cooper, G. G. Borisy, and F. B. Gertler. 2002. Antagonism between Ena/VASP proteins and actin filament capping regulates fibroblast motility. Cell. 109:509-521.
Benz, P. M., C. Blume, S. Seifert, S. Wilhelm, J. Waschke, K. Schuh, F. Gertler, T. Münzel, and T. Renné. 2009. Differential VASP phosphorylation controls remodeling of the actin cytoskeleton. J Cell Sci. 122:3954-3965.
Boëda, B., D. Briggs, T. Higgins, B. Garvalov, A. Fadden, N. McDonald, and M. Way. 2007. Tes, a specific Mena interacting partner, breaks the rules for EVH1 binding. Molecular Cell. 28:1071-1082.
Bulinski, J. C., D. J. Odde, B. J. Howell, T. D. Salmon, and C. M. Waterman-Storer. 2001. Rapid dynamics of the microtubule binding of ensconsin in vivo. J Cell Sci. 114:3885-3897. Calderwood, D. A. 2004. Integrin activation. J Cell Sci. 117:657-666.
Caswell, P. T., M. Chan, A. J. Lindsay, M. W. McCaffrey, D. Boettiger, and J. C. Norman. 2008. Rab-coupling protein coordinates recycling of alpha5beta1 integrin and EGFR1 to promote cell migration in 3D microenvironments. The Journal of Cell Biology. 183:143-155.
Caswell, P. T., S. Vadrevu, and J. C. Norman. 2009. Integrins: masters and slaves of endocytic transport. Nat Rev Mol Cell Biol. 10:843-853.
Chung, J., and T. H. Kim. 2008. Integrin-dependent translational control: Implication in cancer progression. Microsc Res Tech. 71:380-386.
Clark, K., R. Pankov, M. A. Travis, J. A. Askari, A. P. Mould, S. E. Craig, P. Newham, K. M. Yamada, and M. J. Humphries. 2005. A specific alpha5beta1-integrin conformation promotes directional integrin translocation and fibronectin matrix formation. J Cell Sci. 118:291-300.
Comerford, K. M., D. W. Lawrence, K. Synnestvedt, B. P. Levi, and S. P. Colgan. 2002. Role of vasodilator-stimulated phosphoprotein in PKA-induced changes in endothelial junctional permeability. FASEB J. 16:583-585.
Danen, E. H. J., P. Sonneveld, C. Brakebusch, R. Fassler, and A. Sonnenberg. 2002. The fibronectin-binding integrins alpha5beta1 and alphavbeta3 differentially modulate RhoA-GTP loading, organization of cell matrix adhesions, and fibronectin fibrillogenesis. Journal of Cell Biology. 159:1071-1086.
de Hoog, C. L., L. J. Foster, and M. Mann. 2004. RNA and RNA binding proteins participate in early stages of cell spreading through spreading initiation centers. Cell. 117:649-662.
Deevi, R. K., M. Koney-Dash, A. Kissenpfennig, J. A. Johnston, K. Schuh, U. Walter, and K. Dib. 2010. Vasodilator-Stimulated Phosphoprotein Regulates Inside-Out Signaling of 2 Integrins in Neutrophils. The Journal of Immunology. 184:6575-6584.
Defawe, O. D., S. Kim, L. Chen, D. Huang, R. D. Kenagy, T. Renné, U. Walter, G. Daum, and A. W. Clowes. 2010. VASP phosphorylation at serine-239 regulates the effects of NO on smooth muscle cell invasion and contraction of collagen. J. Cell. Physiol. 222:230-237.
Dent, E. W., A. V. Kwiatkowski, L. M. Mebane, U. Philippar, M. Barzik, D. A. Rubinson, S. Gupton, J. E. Van Veen, C. Furman, J. Zhang, A. S. Alberts, S. Mori, and F. B. Gertler. 2007. Filopodia are required for cortical neurite initiation. Nat Cell Biol. 9:1347-1359.
Dominguez, R. 2009. Actin filament nucleation and elongation factors—structure function relationships. Critical Reviews in Biochemistry and Molecular Biology. 44:351-366.
Drees, B., E. Friederich, J. Fradelizi, D. Louvard, M. C. Beckerle, and R. M. Golsteyn. 2000. Characterization of the interaction between zyxin and members of the Ena/vasodilator-stimulated phosphoprotein family of proteins. J Biol. Chem. 275:22503-22511.
Drees, F., and F. B. Gertler. 2008. Ena/VASP: proteins at the tip of the nervous system. Curr Opin Neurobiol. 18:53-59.
Ferron, F., G. Rebowski, S. H. Lee, and R. Dominguez. 2007. Structural basis for the recruitment of profilin-actin complexes during filament elongation by Ena/VASP. EMBO J. 26:4597-4606.
Franco, S. J., and A. Huttenlocher. 2005. Regulating cell migration: calpains make the cut. J Cell Sci. 118:3829-3838.
Friedland, J. C., M. H. Lee, and D. Boettiger. 2009. Mechanically activated integrin switch controls alpha5beta1 function. Science. 323:642-644.
Furman, C., A. L. Sieminski, A. V. Kwiatkowski, D. A. Rubinson, E. Vasile, R. T. Bronson, R. Fässler, and F. B. Gertler. 2007. Ena/VASP is required for endothelial barrier function in vivo. The Journal of Cell Biology. 179:761-775.
Galbraith, C. G., K. M. Yamada, and J. A. Galbraith. 2007. Polymerizing Actin Fibers Position Integrins Primed to Probe for Adhesion Sites. Science. 315:992-995.
Gates, J., J. P. Mahaffey, S. L. Rogers, M. Emerson, E. M. Rogers, S. L. Sottile, D. Van Vactor, F. B. Gertler, and M. Peifer. 2007. Enabled plays key roles in embryonic epithelial morphogenesis in Drosophila. Development. 134:2027-2039.
Geese, M., J. J. Loureiro, J. E. Bear, J. Wehland, F. B. Gertler, and A. S. Sechi. 2002. Contribution of Ena/VASP proteins to intracellular motility of listeria requires phosphorylation and proline-rich core but not F-actin binding or multimerization. Molecular Biology of the Cell. 13:2383-2396.
Geiger, B., and K. M. Yamada. 2011. Molecular architecture and function of matrix adhesions. Cold Spring Harbor Perspect Biol. 3.
Gertler, F., and J. Condeelis. 2011. Metastasis: tumor cells becoming MENAcing. Trends Cell Biol. 21:81-90.
Gertler, F. B., K. Niebuhr, M. Reinhard, J. Wehland, and P. Soriano. 1996. Mena, a relative of VASP and Drosophila Enabled, is implicated in the control of microfilament dynamics. Cell. 87:227-239.
Gorrini, C., F. Loreni, V. Gandin, L. A. Sala, N. Sonenberg, P. C. Marchisio, and S. Biffo. 2005. Fibronectin controls cap-dependent translation through beta1 integrin and eukaryotic initiation factors 4 and 2 coordinated pathways. Proc Natl Acad Sci USA. 102:9200-9205. Goswami, S., U. Philippar, D. Sun, A. Patsialou, J. Avraham, W. Wang, F. Di Modugno, P. Nistico, F. B. Gertler, and J. S. Condeelis. 2009. Identification of invasion specific splice variants of the cytoskeletal protein Mena present in mammary tumor cells during invasion in vivo. Clin Exp Metastasis. 26:153-159.
Gupton, S. L., and F. B. Gertler. 2010. Integrin signaling switches the cytoskeletal and exocytic machinery that drives neuritogenesis. Developmental Cell. 18:725-736.
Hansen, S. D., and R. D. Mullins. 2010. VASP is a processive actin polymerase that requires monomeric actin for barbed end association. The Journal of Cell Biology. 191:571-584.
Hauser, W., K. Knobeloch, M. Eigenthaler, S. Gambaryan, V. Krenn, J. Geiger, M. Glazova, E. Rohde, I. Horak, and U. Walter. 1999. Megakaryocyte hyperplasia and enhanced agonistinduced platelet activation in vasodilator-stimulated phosphoprotein knockout mice. Proceedings of the National Academy of Sciences. 96:8120-8125.
Hoffman, L. M., C. C. Jensen, S. Kloeker, C.-L. A. Wang, M. Yoshigi, and M. C. Beckerle. 2006. Genetic ablation of zyxin causes Mena/VASP mislocalization, increased motility, and deficits in actin remodeling. Journal of Cell Biology. 172:771-782.
Homem, C. C. F., and M. Peifer. 2009. Exploring the roles of diaphanous and enabled activity in shaping the balance between filopodia and lamellipodia. Molecular Biology of the Cell. 20:5138-5155.
Huang, C., Z. Rajfur, N. Yousefi, Z. Chen, K. Jacobson, and M. H. Ginsberg. 2009. Talin phosphorylation by CdkS regulates Smurfl-mediated talin head ubiquitylation and cell migration. Nat Cell Biol. 11:624-630.
Humphries, J. D., A. Byron, M.D. Bass, S. E. Craig, J. W. Pinney, D. Knight, and M. J. Humphries. 2009. Proteomic analysis of integrin-associated complexes identifies RCC2 as a dual regulator of Rac1 and Arf6. Sci Signal. 2:rα51.
Huttenlocher, A., and A. R. Horwitz. 2011. Integrins in cell migration. Cold Spring Harbor Perspect Biol. 3:a005074.
Hynes, R. 2002. Integrins Bidirectional, Allosteric Signaling Machines. Cell. 110:673-687.
Hynes, R. O. 2009. The extracellular matrix: not just pretty fibrils. Science. 326:1216-1219.
Hynes, R. O., and A. Naba. 2012. Overview of the matrisome—an inventory of extracellular matrix constituents and functions. Cold Spring Harbor Perspect Biol. 4.
IIić, D., B. Kovacic, K. Johkura, D. D. Schlaepfer, N. Tomasevie, Q. Han, J.-B. Kim, K. Howerton, C. Baumbusch, N. Ogiwara, D. N. Streblow, J. A. Nelson, P. Dazin, Y. Shino, K. Sasaki, and C. H. Damsky. 2004. FAK promotes organization of fibronectin matrix and fibrillar adhesions. J Cell Sci. 117:177-187.
Ivaska, J., and J. Heino. 2011. Cooperation Between Integrins and Growth Factor Receptors in Signaling and Endocytosis. Annu. Rev. Cell Dev. Biol. 27:291-320.
Kong, F., A. J. Garcia, A. P. Mould, M. J. Humphries, and C. Zhu. 2009. Demonstration of catch bonds between an integrin and its ligand. The Journal of Cell Biology. 185:1275-1284.
Kühnel, K., T. Jarchau, E. Wolf, I. Schlichting, U. Walter, A. Wittinghofer, and S. V. Strelkov. 2004. The VASP tetramerization domain is a right-handed coiled coil based on a 15-residue repeat. Proc Natl Acad Sci USA. 101:17027-17032. Lafuente, E., A. van Puijenbroek, M. Krause, C. Carman, G. Freeman, A. Berezovskaya, E. Constantine, T. Springer, F. Gertler, and V. Boussiotis. 2004. RIAM, an Ena/VASP and Profilin ligand, interacts with Rapl-GTP and mediates Rapl-induced adhesion. Developmental Cell. 7:585-595.
Lanier, L. M., M. A. Gates, W. Witke, A. S. Menzies, A. M. Wehman, J. D. Macklis, D. Kwiatkowski, P. Soriano, and F. B. Gertler. 1999. Mena is required for neurulation and commissure formation. Neuron. 22:313-325.
Laukaitis, C. M., D. J. Webb, K. Donais, and A. F. Horwitz. 2001. Differential dynamics of alpha 5 integrin, paxillin, and alpha-actinin during formation and disassembly of adhesions in migrating cells. Journal of Cell Biology. 153:1427-1440.
Lebrand, C., E. W. Dent, G. A. Strasser, L. M. Lanier, M. Krause, T. M. Svitkina, G. G. Borisy, and F. B. Gertler. 2004. Critical role of Ena/VASP proteins for filopodia formation in neurons and in function downstream of netrin-1. Neuron. 42:37-49.
Lee, H.-S., C. J. Lim, W. Puzon-McLaughlin, S. J. Shattil, and M. H. Ginsberg. 2009. RIAM activates integrins by linking talin to ras GTPase membrane-targeting sequences. J Biol. Chem. 284:5119-5127.
Loureiro, J. J., D. A. Rubinson, J. E. Bear, G. A. Baltus, A. V. Kwiatkowski, and F. B. Gertler. 2002. Critical roles of phosphorylation and actin binding motifs, but not the central proline-rich region, for Ena/vasodilator-stimulated phosphoprotein (VASP) function during cell migration. Molecular Biology of the Cell. 13:2533-2546.
Margadant, C., H. N. Monsuur, J. C. Norman, and A. Sonnenberg. 2011. Mechanisms of integrin activation and trafficking. Current Opinion in Cell Biology. 23:607-614.
McDonnell, A. V., T. Jiang, A. E. Keating, and B. Berger. 2006. Paircoil2: improved prediction of coiled coils from sequence. Bioinformatics. 22:356-358.
Mogilner, A., and G. Oster. 2003. Polymer motors: pushing out the front and pulling up the back. Current Biology. 13:R721-33.
Mogilner, A., and K. Keren. 2009. The shape of motile cells. Curr Biol. 19:R762-71.
Moser, M., K. R. Legate, undefined author, and R. Fassler. 2009. The tail of integrins, talin, and kindlins. Science. 324:895-899.
Muller, P. A. J., P. T. Caswell, B. Doyle, M. P. Iwanicki, E. H. Tan, S. Karim, N. Lukashchuk, D. A. Gillespie, R. L. Ludwig, P. Gosselin, A. Cromer, J. S. Brugge, O. J. Sansom, J. C. Norman, and K. H. Vousden. 2009. Mutant p53 drives invasion by promoting integrin recycling. Cell. 139:1327-1341.
Pankov, R., and A. Momchilova. 2009. Fluorescent labeling techniques for investigation of fibronectin fibrillogenesis (labeling fibronectin fibrillogenesis). Methods Mol. Biol. 522:261-274.
Pankov, R., E. Cukierman, B. Z. Katz, K. Matsumoto, D. C. Lin, S. Lin, C. Hahn, and K. M. Yamada. 2000. Integrin dynamics and matrix assembly: tensin-dependent translocation of alpha(5)beta(1) integrins promotes early fibronectin fibrillogenesis. Journal of Cell Biology. 148:1075-1090.
Philippar, U., E. T. Roussos, M. Oser, H. Yamaguchi, H.-D. Kim, S. Giampieri, Y. Wang, S. Goswami, J. B. Wyckoff, D. A. Lauffenburger, E. Sahai, J. S. Condeelis, and F. B. Gertler. 2008. A Mena invasion isoform potentiates EGF-induced carcinoma cell invasion and metastasis. Developmental Cell. 15:813-828.
Pula, G., and M. Krause. 2008. Role of Ena/VASP proteins in homeostasis and disease. Handbook of experimental pharmacology. 39.
Rantala, J. K., J. Pouwels, T. Pellinen, S. Veltel, P. Laasola, E. Mattila, C. S. Potter, T. Duffy, J. P. Sundberg, O. Kallioniemi, J. A. Askari, M. J. Humphries, M. Parsons, M. Salmi, and J. Ivaska. 2011. SHARPIN is an endogenous inhibitor of β1-integrin activation. Nat Cell Biol. 13:1315-1324.
Robinson, B. D., G. L. Sica, Y.-F. Liu, T. E. Rohan, F. B. Gertler, J. S. Condeelis, and J. G. Jones. 2009. Tumor microenvironment of metastasis in human breast carcinoma: a potential prognostic marker linked to hematogenous dissemination. Clin Cancer Res. 15:2433-2441.
Roussos, E. T., M. Balsamo, S. K. Alford, J. B. Wyckoff, B. Gligorijevic, Y. Wang, M. Pozzuto, R. Stobezki, S. Goswami, J. E. Segall, D. A. Lauffenburger, A. R. Bresnick, F. B. Gertler, and J. S. Condeelis. 2011a. Mena invasive (MenaINV) promotes multicellular streaming motility and transendothelial migration in a mouse model of breast cancer. J Cell Sci. 124:2120-2131.
Roussos, E. T., S. Goswami, M. Balsamo, Y. Wang, R. Stobezki, E. Adler, B. D. Robinson, J. G. Jones, F. B. Gertler, J. S. Condeelis, and M. H. Oktay. 2011b. Mena invasive (Mena(INV)) and Mena11a isoforms play distinct roles in breast cancer cell cohesion and association with TMEM. Clin Exp Metastasis. 28:515-527.
Roussos, E. T., Y. Wang, J. B. Wyckoff, R. S. Sellers, W. Wang, J. Li, J. W. Pollard, F. B. Gertler, and J. S. Condeelis. 2010. Mena deficiency delays tumor progression and decreases metastasis in polyoma middle-T transgenic mouse mammary tumors. Breast Cancer Res. 12:R101.
Schwarzbauer, J. E., and D. W. DeSimone. 2011. Fibronectins, Their Fibrillogenesis, and In Vivo Functions. Cold Spring Harbor Perspect Biol. 3:a005041-a005041.
Shapiro, I. M., A. W. Cheng, N. C. Flytzanis, M. Balsamo, J. S. Condeelis, M. H. Oktay, C. B. Burge, and F. B. Gertler. 2011. An EMT-Driven Alternative Splicing Program Occurs in Human Breast Cancer and Modulates Cellular Phenotype. PLoS Genet. 7:e1002218.
Singh, P., C. Carraher, and J. E. Schwarzbauer. 2010. Assembly of fibronectin extracellular matrix. Annu. Rev. Cell Dev. Biol. 26:397-419.
Stanchi, F., C. Grashoff, C. F. Nguemeni Yonga, D. Grail, R. Fassler, and E. Van Obberghen-Schilling. 2009. Molecular dissection of the ILK-PINCH-parvin triad reveals a fundamental role for the ILK kinase domain in the late stages of focal-adhesion maturation. J Cell Sci. 122:1800-1811.
Tuomi, S., A. Mai, J. Nevo, J. O. Laine, V. Vilkki, T. J. Ohman, C. G. Gahmberg, P. J. Parker, and J. Ivaska. 2009. PKCepsilon regulation of an alpha5 integrin-ZO-1 complex controls lamellae formation in migrating cancer cells. Sci Signal. 2:ra32.
Toyoda, A., A. Yokota, T. Saito, H. Kawana, M. Higashi, Y. Suzuki, T. Tanaka, M. Kitagawa, and K. Harigaya. 2011. Overexpression of human ortholog of mammalian enabled (hMena) is associated with the expression of mutant p53 protein in human breast cancers. Int. J. Oncol. 38:89-96.
Valastyan, S., N. Benaich, A. Chang, F. Reinhardt, and R. A. Weinberg. 2009. Concomitant suppression of three target genes can explain the impact of a microRNA on metastasis. Genes Dev. 23:2592-2597.
van der Flier, A., K. Badu-Nkansah, C. A. Whittaker, D. Crowley, R. T. Bronson, A. Lacy-Hulbert, and R. O. Hynes. 2010. Endothelial alpha5 and alphav integrins cooperate in remodeling of the vasculature during development. Development. 137:2439-2449.
Vicente-Manzanares, M., and A. R. Horwitz. 2011. Adhesion dynamics at a glance. J Cell Sci. 124:3923-3927.
Vouret-Craviari, V., E. Boulter, D. Grall, C. Matthews, and E. Van Obberghen-Schilling. 2004. ILK is required for the assembly of matrix-forming adhesions and capillary morphogenesis in endothelial cells. J Cell Sci. 117:4559-4569.
Warzecha, C. C., T. K. Sato, B. Nabet, J. B. Hogenesch, and R. P. Carstens. 2009. ESRP1 and ESRP2 are epithelial cell-type-specific regulators of FGFR2 splicing. Molecular Cell. 33:591-601.
Whittaker, C. A., K.-F. Bergeron, J. Whittle, B. P. Brandhorst, R. D. Burke, and R. O. Hynes. 2006. The echinoderm adhesome. Developmental Biology. 300:252-266.
Wickström, S. A., K. Radovanac, and R. Fassler. 2011. Genetic analyses of integrin signaling. Cold Spring Harbor Perspect Biol. 3.
Worth, D. C., K. Hodivala-Dilke, S. D. Robinson, S. J. King, P. E. Morton, F. B. Gertler, M. J. Humphries, and M. Parsons. 2010. Alpha v beta3 integrin spatially regulates VASP and RIAM to control adhesion dynamics and migration. The Journal of Cell Biology. 189:369-383.

Yang, J. T., B. L. Bader, J. A. Kreidberg, M. Ullman-Culleré, J. E. Trevithick, and R. O. Hynes. 1999. Overlapping and independent functions of fibronectin receptor integrins in early mesodermal development. Developmental Biology. 215:264-277.
Zaidel-Bar, R., C. Ballestrem, Z. Kam, and B. Geiger. 2003. Early molecular events in the assembly of matrix adhesions at the leading edge of migrating cells. J Cell Sci. 116:4605-4613.
Zaidel-Bar, R., S. Itzkovitz, A. Ma'ayan, R. Iyengar, and B. Geiger. 2007. Functional atlas of the integrin adhesome. Nat Cell Biol. 9:858-867.
Zamir, E., M. Katz, Y. Posen, N. Erez, K. M. Yamada, B. Z. Katz, S. Lin, D. C. Lin, A. Bershadsky, Z. Kam, and B. Geiger. 2000. Dynamics and segregation of cell-matrix adhesions in cultured fibroblasts. Nat Cell Biol. 2:191-196.
Zhang, X., G. Jiang, Y. Cai, S. J. Monkley, D. R. Critchley, and M. P. Sheetz. 2008. Talin depletion reveals independence of initial cell spreading from integrin activation and traction. Nat Cell Biol. 10:1062-1068.
Zimmermann, J., D. Labudde, T. Jarchau, U. Walter, H. Oschkinat, and L. J. Ball. 2002. Relaxation, equilibrium oligomerization, and molecular symmetry of the VASP (336-380) EVH2 tetramer. Biochemistry. 41:11143-11151.

1. A method of treating invasion of a tumor in a subject or inhibiting metastasis of a tumor in a subject comprising administering to the subject an agent which inhibits the interaction of a Mena with an alpha5 integrin in an amount effective to treat invasion or inhibit metastasis of a tumor.

2. A method of treating a fibronectin deposition disease in a subject or a fibroproliferative disease in a subject comprising administering to the subject an agent which inhibits the interaction of a Mena with an alpha5 integrin in an amount effective to treat fibronectin deposition or fibroproliferative disease.

3. The method of claim 1, wherein the agent inhibits the interaction of Mena with the C-terminal 5 residues of an alpha5 integrin C-terminal cytoplasmic tail.

4. The method of claim 1, wherein the agent inhibits the interaction of a LERER repeat region of Mena with an alpha5 integrin.

5. The method of claim 1, wherein the tumor is a breast cancer tumor.

6. The method of claim 1, wherein the alpha5 integrin is part of an alpha5 beta1 integrin complex.

7. The method of claim 6, wherein the alpha5 beta1 integrin is a fibronectin receptor.

8. The method of claim 1, wherein the agent is a small organic molecule, an antibody, a fragment of an antibody, a peptide or an oligonucleotide aptamer.

9. The method of claim 1, wherein the agent competes for binding to the alpha5 integrin with a LERER repeat region of a Mena.

10. The method of claim 1, wherein the Mena is a human Mena.

11. The method of claim 1, wherein the Mena is a Mena^{INV}.

12. (canceled)

13. A method for identifying an agent as an inhibitor of an interaction of Mena with an alpha5 integrin, the method comprising contacting the alpha5 integrin with Mena (a) in the presence of and (b) in the absence of the agent under conditions permitting Mena to interact with the alpha5 integrin and quantifying the interaction of Mena with the alpha5 integrin in the presence and in the absence of the agent, and identifying the agent as an inhibitor or not of an interaction of Mena with an alpha5 integrin, wherein quantification of a decreased interaction of Mena with the alpha5 integrin in the presence of the agent compared to in the absence of the agent indicates that the agent is an inhibitor of the interaction of Mena with the alpha5 integrin, and wherein quantification of no change in interaction, or an increased interaction, of Mena with the alpha5 integrin in the presence of the agent compared to in the absence of the agent indicates that the agent is not an inhibitor of the interaction of Mena with the alpha5 integrin.

14. The method of claim 13, wherein quantifying the interaction of Mena with the alpha5 integrin in the presence of and in the absence of the agent comprises quantifying the amount of Mena bound to alpha5 integrin.

15. The method of claim 13, wherein quantifying the interaction of Mena with the alpha5 integrin in the presence and in the absence of the agent comprises quantifying the activity of alpha5 integrin.

16. The method of claim 13, wherein the alpha5 integrin is part of an alpha5 beta1 integrin complex.

17. The method of claim 13, wherein the agent is a small organic molecule, an antibody, a fragment of an antibody, a peptide or an oligonucleotide aptamer.

18. The method of claim 13, wherein the agent inhibits the interaction of Mena with the C-terminal 5 residues of the alpha5 integrin C-terminal cytoplasmic tail.

19. The method of claim 13, wherein the agent competes for binding to the alpha5 integrin with a LERER repeat region of Mena.

20. The method of claim 13, wherein the Mena is a human Mena.

21. The method of claim 13, wherein the Mena is a Mena^{INV}.

22. (canceled)
